(19) United States
(12) Patent Application Publication
(10) Pub. No.: US 2003/0077826 A1
(43) Pub. Date: Apr. 24, 2003

(54) Chimeric molecules containing a module able to target specific cells and a module regulating the apoptogenic function of the permeability transition pore complex (PTPC)

(76) Inventors: Lena Edelman, Boulogne (FR); Etienne Daniel Francois Jacotot, Paris (FR); Jean-Paul Briand, Strasbourg (FR)

Correspondence Address: FINNEGAN, HENDERSON, FARABOW,, GARRETT & DUNNER, L.L.P., 1300 I Street, NW, Washington, DC, 20005-3315, US

(21) Appl. No.: 10/059,261

(22) Filed: Jan. 31, 2002

Related U.S. Application Data
(60) Provisional application No. 60/265,594, filed on Feb. 2, 2001.

(51) Int. Cl. A61K 39/395; C12N 15/00

(52) U.S. Cl. 435/440; 424/146.1; 424/155.1

(57) ABSTRACT

A chimeric polypeptide has the formula: pTox-pTarg, wherein pTox is a viral apoptotic peptide, such as the Vpr peptide of HIV-1 or a fragment of the Vpr peptide of HIV-1 containing the amino acid motif H(F/S)RIG that interacts with mitochondrial inner membrane, adenine nucleotide translocation (ANT) protein of a cell. pTarg is an antibody or an antibody fragment that binds to the outer membrane of the cell. Binding of the chimeric polypeptide to the cell is followed by apoptosis of the cell. A vector encoding a chimeric polypeptide and a recombinant host cell comprising the vector are provided. The chimeric polypeptide is useful for targeting pTox to cells, such as cancer cells.

### CROSS-REFERENCE TO RELATED APPLICATIONS

The application hereby claims the benefit under 35 U.S.C. § 119(e) of U.S. provisional application Serial No. 60/265,594, filed Feb. 2, 2001. The entire disclosure of this application is relied upon and incorporated by reference herein.

### BACKGROUND OF THE INVENTION

1. Field of the Invention

The present invention relates generally to cell death regulatory molecules for therapeutic use. More specifically, this invention relates to molecules in which a peptidic or pseudo-peptidic part acting on the permeability transition pore complex (PTPC) is covalently linked to cell-targeting molecules including antibodies, recombinant antibody fragments or homing peptides. The resulting chimeric molecules are polypeptides or peptidomimetic molecules which target the PTPC and/or its major component the adenine nucleotide translocation (ANT) to induce or inhibit cell death (apoptosis). This invention also relates to such chimeric molecules when the PTPC-interacting part is an apoptogenic HIV-1 Vpr-derived peptide (or pseudopeptide) or an ANT-derived peptide (or pseudo-peptide). This invention also relates to nucleic acid sequence construct encoding such chimeric molecule or encoding portions of these chimeric molecules.

2. Background

It is currently agreed that mitochondria play an important role in controlling life and death of cells (apoptosis; Kroemer and Reed 2000, Nature Medicine). It appears both that an increasing number of molecules involved in the transduction of the signal and also many metabolites and certain viral effectors act on mitochondria and influence the permeabilisation of mitochondrial membranes. Using mitochondrial-specific pro-apoptotic agent would seem to be an emerging concept in cancer therapy (Costantini et al 2000, Journal of the National Cancer Institute). Similarly, it might be possible to use cytoprotective molecules, thanks to their ability to stabilize mitochondrial membranes, in the treatment of illnesses where there is excessive apoptosis (neurodegenerative diseases, ischemia, AIDS, fulminant hepatitis, etc.).

Mitochondrial membrane permeabilisation (MMP) is a key event of apoptotic cell death associated with the release of caspase activators and caspase-independent death effectors from the intermembrane space, dissipation of the inner transmembrane potential (ΔΨm), as well as a perturbation of oxidative phosphorylation (Green and Reed, 1998; Gross et al., 1999; Kroemer and Reed, 2000; Kroemer et al., 1997; Lemasters et al., 1998; Vander Heiden and Thompson, 1999; Wallace, 1999). Pro- and anti-apoptotic members of the Bcl-2 family regulate inner and outer MMP through interactions with the adenine nucleotide translocation (ANT; in the inner membrane, IM), the voltage-dependent anion channel (VDAC; in the outer membrane, OM), and/or through autonomous channel-forming activities (Desagher et al., 1999; Gross et al., 1999; Kroemer and Reed, 2000; Marzo et al., 1998; Shimizu et al., 1999; Vander Heiden and Thompson, 1999). ANT and VDAC are major components of the permeability transition pore complex (PTPC), a polyprotein structure organized at sites at which the two mitochondrial membranes are apposed (Crompton, 1999; Kroemer and Reed, 2000).

The mitochondrial phase is under the control of Bcl-2 family of oncogenes and anti-oncogenes (for review: 5; 28) involved in more than 50% of cancers (29). All members of Bcl-2 family play an active role in the regulation of apoptosis, some of them being proapoptotic (Bax, Bak, Bcl-X_{S}, Bad, etc.) and others, being antiapoptotic (Bcl-2, Bcl-X_{L}, Bcl-w, Mcl-1, etc.) (G. Kroemer, Nat Med 3, 614-20 (1997)).

The mitochondrial megachannel is a polyprotein complex formed in the contact site between the inner and the outer mitochondrial membranes that participate in the regulation of mitochondrial membrane permeability. It is composed of a set of proteins including mitochondrion-associated hexokinase (HK), porin (voltage-dependent anion channel or VDAC), adenine nucleotide translocation (ANT), peripheral benzodiazepin receptor (PBR), creatine kinase (CK), and cyclophilin D, as well as Bcl-2 family members. In physiological conditions, PTPC controls the mitochondrial calcium homeostasis via the regulation of its conductance by the mitochondrial pH, the ΔΨm, NAD/NAD(P)H redox equilibrium and matrix protein thiol oxidation. (M. Zoratti, I. Szabo, Biochim, Biophys Acta 1241, 139-76 (1995). S. Shimizu, M. Narita, Y. Tsujimoto, Nature 399, 483-487 (1999). M. Crompton, Biochem J 341, 233-249 (1999). K. Woodfield, A. Ruck, D. Brdiczka, A. P. Halestrap, Biochem J 336, 287-90 (1998). P. Bernardi, K. M. Broekemeier, D. R. Pfeiffer, J Bioenerg Biomembr 26, 509-17 (1994). F. Ichas, L. Jouaville, J. Mazat, Cell 89, 1145-53 (1997)).

Apoptosis and related forms of controlled cell death are involved in a great number of illness. Excess or insufficiency of cell death processes are involved in auto-immune and neurodegenerative diseases, cancers, ischemia, and pathological infections or diseases such as viral and bacterial infections. Just few examples illustrating the virtually ubiquitous involvement of mitochondria in diseases associated with the abnormal control of cell death will be mentioned here.

In different models of ischemia (heart, liver, kidney or brain), using molecules that are capable of stabilising mitochondrial membranes, such as CsA for example (or its analogous non-immunosuppressor-Me-Val4-CsA) has made it possible to reduce massive apoptosis and its acute consequences at the level of the organ. In addition, VDAC is indispensable for the destruction of neurons of the rat hippocampus after hypoxic reperfusion. In the area of neurodegenerative diseases, a great many observations suggest close links with mitochondrial control of apoptosis (see Kroemer and Reed 2000, Nature Medicine). The neurotoxin-methyl-4-phenylpyridinium induces mitochondrial permeability transition and the exit of cytochrome c. Poisoning by mitochondrial toxins such as nitro-propionic acid or rotenone provokes in primates and rodents a Huntington-disease type of illness.

PTPC is a dynamic protein complex located at the contact site between the two mitochondrial membranes, its opening allowing the free diffusion of solutes <1500 Da on the inner membrane. Formation of PTPC involves the association of proteins from different compartments, hexokinase (cytosol), porin, also called voltage-dependent anion channel (VDAC, outer membrane), peripheral benzodiazepin receptor (PBR, outer membrane), ANT (inner membrane) and cyclophilin D (matrix). PTPC has been implicated in many examples of apoptosis due to its capacity to integrate multiple pro-apoptotic signal transduction pathways and due to its control by proteins from Bcl-2/Bax family. The Bcl-2 family comprises death inhibitory (Bcl-2-like) and death inducing (Bax-like) members which respectively prevent or facilitate PTPC opening. Bax and Bcl-2 reportedly interact with VDAC and ANT within PTPC. In physiological conditions, ANT is a specific antiporter for ADP and ATP. However, ANT can also form a lethal pore upon interaction with different pro-apoptotic agents. including Ca2+, atractyloside, HIV-1 Vpr-derived peptides and pro-oxidants. Mitochondrial membrane permeabilization may also be regulated by the non-specific VDAC pore modulated by Bcl-2/Bax-like proteins in the outer membrane (12; 16). and/or by changes in the metabolic ATP/ADP gradient between the mitochondrial matrix and the cytoplasm (17).

There is a need in the art for cytoprotective molecules in ischemia, neurodegenerative diseases, fulminant hepatitis and viral infections.

Another application of the chimeric molecule according the invention can be contemplated for the preparation of cosmetics or for preventing early death of plants or vegetables or flowers particularly for preventing the opening of the PTPC.

Conventional chemotherapeutic agents are limited in their therapeutic effectiveness by severe side effects due to their poor selectivity for tumors. The development of monoclonal antibodies (and ScFv) against specific tumor antigens and the identification of homing peptides specific for tumor vascularisation have made it possible to consider enhancing the selectivity of anticancer drugs by a targeted delivery approach. However, such reported attempts using monoclonal antibodies and the anticancer drugs doxorubicin (Trail P. A., et al 1993 Science 261:212), metotrexate (Kanellos J. et al., 1985 J Natl Cancer Inst 75:319), and Vinca alkaloids (Starling J. J. et al., 1991 Cancer Res 41:2965), have been largely unsuccessful. These antibody-drug conjugates were only moderately potent and usually less cytotoxic than the corresponding unconjugated drugs. In fact, antigen-specific cytotoxicity toward cultured tumor cells was rarely demonstrated. In vivo therapeutic effects with these conjugates in tumor zenograft animal models were in general observed only when the treatments were commenced before the tumors were well established or when exceedingly large doses (up to 90 mg/kg, drug equivalent dse) were used. It is, therefore, not surprising that in human clinical trials, no significant antitumor effects were observed with these agents (Elias D. J. et al., 1994 Am Respir Crit Care Med 150:1114) (Schneck D. et al., 1990). Indeed, the peak circulating serum concentrations of conjugates were only in the same range as their in vitro IC50 value and thus, capable of eliminating at best only about 50% of tumor cells.

These observations led to the conclusion that the previous attempts at delivering therapeutic doses of cytotoxic drugs via monoclonal antibodies have met with little success in clinical trials because of inappropriate choice of drug. One possible (partial-) solution was to conclude that immunoconjugates must be composed of drugs possessing much higher potency than the clinically used anticancer agents if therapeutic levels of conjugate at the tumor sites are to be achieved in patients. Effectively, such toxins, including maytansinoides, enediynes, or intercalating agents CC1065, were shown to be 100 to 1000-fold more cyctotoxic than the chemotherapeutic agents doxorubicin, methotrexate, and Vinca alkaloids (Chari R V J et al., 1995 Cancer Res 55:4079) (Chari R V J et al., 1992, Cancer Res 52:127).

Another approach termed “Adept” was also designed. This antibody-directed enzyme prodrug therapy (Adept) is based upon the use of a monoclonal antibody to target an enzyme at the tumor cell surface, which ultimately is expected to selectively deliver an antitumor drug from a suitable inactive prodrug. In both cases, clinical trials are in progress; however, since today none of them have been introduced in cancer chemotherapy, there is a need for new tools to kill target tumor cells. Bagshawe K D, 1990. Antibody-directed enzyme/prodrug therapy (ADEPT). Biochem Soc Trans. 18(5):750-2. Melton R G, Sherwood R F. 1996 Antibody-enzyme conjugates for cancer therapy. J Natl Cancer Inst, 88(3-4):153-65. Rihova B. 1997; Targeting of drugs to cell surface receptors. Crit Rev Biotechnol. 17(2):149-69. Hudson P J. 2000. Recombinant antibodies: a novel approach to cancer diagnosis and therapy. Expert Opin Investig Drugs 9(6):1231-42.

Recently, the mitochondrion has been proposed as a novel prospective target for chemotherapy-induced apoptosis (1-7). Indeed, four different anti-cancer agents, including the resinoid acid-derivative CD437, lonidamine, betulinic acid, and arsenite, have been shown to induce cancer cell apoptosis by a direct action on mitochondria. The interaction of these anti-cancer agents with mitochondria results in an increase of the permeability of the inner mitochondrial membrane due, at least in part, to the opening of the permeability transition pore complex (PTPC). PTPC opening leads to swelling of the mitochondria matrix, the dissipation of the inner transmembrane potential (ΔΨm), enhanced generation of reactive oxygen species (ROS), and the release of apoptogenic proteins from the intermembrane space to the cytoplasm. Such mitochondrial apoptogenic effectors include the caspase activator cytochrome c, apoptosis inducing factor (AIF), and pro-caspases (2-6). All the signs of apoptosis induced by CD437, lonidamine, betulinic acid, and arsenite are prevented by two agents acting on specific PTPC proteins, namely cyclopsporin A (CsA, a cyclophilin D ligand) and bongkrekic acid (BA, a ligand of the adenine nucleotide translocase (ANT)). It thus appears that PTPC opening is a critical event of apoptosis triggered by these agents.

Mastoparan, a peptide isolated from wasp venom, is the first peptide known to induce mitochondrial membrane permeabilization via a CsA-inhibitable mechanism and to induce apoptosis via a mitochondrial effect when added to intact cells. This peptide has an α-helical structure and possesses some positive charges that are distributed on one side of the helix. A similar peptide (KLAKLAKKLAKLAK or (KLAKLAK)₂ (K=lysine, L=alanine, and A=leucine) has been found recently to disrupt mitochondrial membranes when it is added to purified mitochondria, although the mechanisms of this effect have not been elucidated.

The vasculature of individual tissues is highly specialized. The endothelium in lymphoid tissues expresses tissue-specific receptors for lymphocyte homing, and recent work utilizing phage homing has revealed an unprecedented degree of specialization in the vasculature of other normal tissues. In vivo screening of libraries of phage that displace random peptide sequences on their surfaces has yielded specific homing peptides for a large number of normal tissues. The tissue-specific endothelial molecules to which the phage peptides home may serve as receptors for metastasizing malignant cells. Probing of tumor vasculature has yielded peptides that home to endothelial receptors expressed selectively in angiogenic neovasculature. These receptors, and those specific for the vasculature of individual normal tissues, are likely to be useful in targeting therapies to specific sites. Ruoslahti E, Rajotte D. 2000; An address system in the vasculature of normal tissues and tumors. Annu Rev Immunol. 18:813-27.

Ellerby et al. recently have fused the mitochondriotoxic (KLAKLAK)₂ motif to a targeting peptide that interacts with endothelial cells. Such a fusion peptide is internalized and induces mitochondrial membrane permeabilization in angiogenicendothelial cells and kills MDA-MD-435 breast cancer xenografts transplanted into nude mice. Similarly, a recombinant chimeric protein containing interleukin 2 (IL-2) protein fused to Bax selectively binds to and kills IL-2 receptor-bearing cells in vitro. Thus, specific cytotoxic agents that target surface receptors, translocate into the cytoplasm, and induce apoptosis via mitochondrial membrane permeabilization might be useful in treating cancer.

There is a need in the art for the selective eradication of transformed cells. One strategy is to target a toxic agent to selected cell types. More particularly, there exists a need in the art for method and reagents for regulating mitochondrial permeabilization and apoptosis.

### SUMMARY OF THE INVENTION

In order to overcome at least some of the limitations of the prior art, the present invention provides a peptidic or pseudo-peptidic family of polyfunctional molecules containing a cell-targeting part (termed TARG), a PTPC-interacting part (termed TOX/SAVE), and a facultative mitochondrial localisation sequence (MLS). In a preferred embodiment of the invention, the TOX/SAVE portion of the said polyfunctional molecule is a peptide or peptidomimetic molecule which interact directly with the Adenine Nucleotide Translocator (ANT) a central component of the PTPC

Thus, the present invention includes two categories of targeted cell death regulatory molecules:

TARG-(MLS)-TOX is a polyfunctional molecule which induces a PTPC-dependent mitochondrial membrane permeabilisation and consequent cell death.

TARG-(MLS)-SAVE is a polyfunctional molecule which protects cells from mitochondrial membrane permeabilisation and consequently from cell death through interaction with the PTPC and/or ANT.

The invention further provides a vector encoding a chimeric polypeptide of the invention.

Also, the invention provides a recombinant host cell comprising a vector of the invention.

Further, the invention provides a cancer cell having a tumor-associated antigen on the surface thereof to which the chimeric polypeptide of the invention is bound via the antibody or antibody fragment of the chimeric polypeptide. The invention also provides methods for detecting cancer cells.

The invention also provides methods for inducing or preventing apoptosis with polypeptides of the invention. The invention provides methods for inducing apoptosis in tumor cells. The invention provides methods for inducing apoptosis in virus infected cells.

The invention further provides hybridomas producing polypeptides of the invention. The invention also provides monoclonal antibodies produced by these hybridomas.

The invention also provides methods for identifying active agents of interest that interact with the PTPC. The invention also provides methods for identifying active agents of interest that interact with ANT peptide. The invention also provides methods for identifying mitochondrial antigens.

The invention also provides methods of treatment or prevention of a pathological infection or disease by administering a polypeptide of the invention to a patient. The invention also provides pharmaceutical compositions comprising a polypeptide of the invention.

### BRIEF DESCRIPTION OF THE DRAWINGS

FIG. 1 shows the nucleotide sequence of vector pACgp67-ScFv461.

FIG. 2 shows the nucleotide sequence of vector pACgp67-ScFv350.

FIG. 3 shows the nucleotide sequence of Vh and VL, from the clone therap 99B3.

FIG. 4 shows the nucleotide sequence of Vh and VL from the clone therap.88E10.

FIG. 5 shows the nucleotide sequence of Vh and VL from the clone therap.152C3.

FIGS. 6, 7, 8, 9, 10, 11 show surface plasmon resonance curves.

FIGS. 12 and 13 show the strategy for obtaining the ScFv-transfert vector.

### DETAILED DESCRIPTION OF THE INVENTION

It was recently discovered that the proapoptotic HIV-1-encoded protein Vpr induces mitochondrial membrane permeabilization via its physical and functional interaction with the mitochondrial inner membrane protein ANT (adenine nucleotide translocation, also called ADP/ATP carrier). This was shown using a variety of different techniques: surface plasmon resonance, electrophysiology, synthetic proteoliposomes, studies on purified mitochondria (respirometry, electron microscopy, organellofluorometry), as well as microinjection of intact cells. These discoveries are described in detail in U.S. Provisional Application No. 60/231,539 filed Sep. 11, 2000, the entire disclosure of which is relied upon and incorporated by reference herein.

The present invention pertains to novel cytotoxic conjugates based on the association between a peptidic molecule (named pTox) interacting with the mitochondrial permeability transition pore complex (PTPC) and a molecule (named pTarg) able to target cells. The present invention also pertains to novel cytoprotective conjugates based on the association between a peptidic molecule (named SAVE) interacting with the mitochondrial permeability transition pore complex (PTPC) and a molecule (named pTarg) able to target the cells to rescue. In a specific embodiment of this invention, a cytotoxic conjugate of the invention includes a viral derived pro-apoptotic peptide.

In one embodiment of the invention, the polyfunctional molecule TARG-(MLS)-TOX is a tumor specific molecule that selectively interact with a tumor cell or a specific mammalian cell type, where the polyfunctional molecule is selectively internalised by the mammalian or tumoral cell type, where the polyfunctional molecule interact with the PTPC and/or ANT and exhibits thereto a strong mitochondrio-toxicity leading to apoptosis or any cell death process.

In one embodiment of the invention, the polyfunctional molecule TARG-(MLS)-TOX exhibits a selective toxicity against angiogenic endothelial cells. In another embodiment of the invention, the polyfunctional molecule TARG-(MLS)-TOX exhibits a selective toxicity against tumor cells.

In one embodiment of the invention, the TARG part of the polyfunctional molecule TARG-(MLS)-TOX is an antibody or a recombinant antibody fragment. In another embodiment of the invention, the TARG part of the polyfunctional molecule TARG-(MLS)-TOX is tumor horning peptide (example; CNGRC peptide; lung-homing peptide CGFECVRQCPERC).

In one embodiment of the invention, the TOX part of the polyfunctional molecule TARG-(MLS)-TOX is a peptide or a peptido-mimetic derived from the C-terminal part (amino-acids 52 to 96) of the HIV-1 Vpr protein.

In one embodiment of the invention, the TOX part of the polyfunctional molecule TARG-(MLS)-TOX is a pro-apoptotic Bcl-2 family member such as the Bax or Bid proteins, or a fragment thereof.

In one embodiment of the invention, the TOX part of the polyfunctional molecule TARG-(MLS)-TOX is a D-peptide, is a Ψ-peptide or a retro-inverso peptide chosen among the group of peptidic sequences described in table 1:

TABLE I

| Name | TOX Peptidic Sequences |
|---|---|
| Vpr71-82 | HFRIGCRHSRIG |
| Vpr71-82[R73,77,80K] | HFKIGCKHSKIG |
| Vpr71-96 | HFRIGCRHSRIGIIQQRRTRNGASKS |
| Vpr71-96[R73,77,80K] | HFKIGCKHSKIGIIQQRRTRNGASKS |
| Vpr52-96 | DTWTGVEALIRILQQLLFIHFRIGCRHSRIGIIQQRRTRNGASKS |
| Vpr52-96[R73,77,80K] | DTWTGVEALIRILQQLLFIHFKIGCKHSKIGIIQQRRTRNGASKS |
| Vpr52-96[L60,67A] | DTWTGVEAAIRILQQALFIHFRIGCRHSRIGIIQQRRTRNGASKS |
| Vpr52-82 | DTWTGVEALIRILQQLLFIHFRIGCRHSRIG |
| Vpr52-82[R73,77,80K] | DTWTGVEALIRILQQLLFIHFKIGCKHSKIG |
| Histatin5 | DSHARKRHHGYKRKFHEKHHSHRGY |
| Candida Albicans | |
| Mastoparan | INLKALAALAKKIL |
| Vespula Lewisii | |
| hNUR77(555-568) | LSRLLGKLPELRTL |
| hNTR(368-381) | ATLDALLAALRRIQ |
| neutrotrophin receptor | |
| Bid(84-100) | RNIARHLAQVGDSMRDR |
| Bax(57-72) | KKLSECLKRIGDELDS |
| Bax(72-87) | GQVGRQLAIIGDDINR |
| HBX(70-78) | ALRFTSARR |
| DCC(1376-1390) | KTHVKTASLGLAGKA |
| ANT₁(104-116) | DRHKQFWRYFAGN |
| ANT₂(104-116) | DKRTQFWRYFAGN |
| ANT₃(104-116) | DKHTQFWRYFAGN |
| ANT₁(104-116 [A114P] | DRHKQFWRYFPGN |
| ANT₂(104-116)[A114P] | DKRTQFWRYFPGN |
| ANT₃(104-116)[A114P] | DKHTQFWRYFPGN |
| ANT_{1,2,3}(117-134) | LASGGAAGATSLCFVYPL |
| ANT₁(104-134) | DRHKQFWRYFAGNLASGGAAGATSLCFVYPL |
| ANT₂(104-134) | DKRTQFWRYFAGNLASGGAAGATSLCFVYPL |
| ANT₃(104-134) | DKHTQFWRYFAGNLASGGAAGATSLCFVYPL |
| ANT₁(104-134)[A114P] | DRHKQFWRYFPGNLASGGAAGATSLCFVYPL |
| ANT₂(104-134 [A114P] | DKRTQFWRYFPGNLASGGAAGATSLCFVYPL |
| ANT₃(104-134) [A114P] | DKHTQFWRYFPGNLASGGAAGATSLCFVYPL |
| Vpr52-96 [C76S] | DTWTGVEALIRILQQLLFIHFRIGSRHSRIGIIQQRRTRNGASKS |
| HTLV-Ip13II | ₁₉PSLRVWRLCARRLV₃₂ |
| Bad103-127 | NLWAAQRYGRELRRMSDEFVDSFKK |
| Bax52-76 | QDASTKKLSECLKRIGDELDSNMEL |

In one embodiment of the invention, the SAVE part of the polyfunctional molecule TARG-(MLS)-SAVE is a L-peptide, a D-peptide or a retro-inverso peptide chosen among the group of peptidic sequences described in table II:

| Name | SAVE Peptidic Sequences |
|---|---|
| ANT₁(104-116) | DRHKQFWRYFAGN |
| ANT₂(104-116) | DKRTQFWRYFAGN |
| ANT₃(104-116) | DKHTQFWRYFAGN |
| ANT_{1,2,3}(117-134) | LASGGAAGATSLCFVYPL |
| ANT₁(104-134) | DRHKQFWRYFAGNLASGGAAGATSLCFVYPL |
| ANT₂(104-134) | DKRTQFWRYFAGNLASGGAAGATSLCFVYPL |
| ANT₃(104-134) | DKHTQFWRYFAGNLASGGAAGATSLCFVYPL |

In one embodiment of the invention, the TARG part of the polyfunctional molecule TARG-(MIS)-SAVE is a L-peptide, a D-peptide or a retro-inverso peptide chosen among the group of peptidic sequences described in table III:

| | |
|---|---|
| ANTENNAPEDIA | RQIKITFQNRRMKTKK |
| third helix (residues 43- | |
| 58) | |
| HIV-1 Vpr83-96 | IIQQRRTRNGASKS |
| transduction domain | |
| HIV-1 Tat48-59 | GRKKRRQRRRPP |
| transduction domain | |
| HIV-1 Tat49-57 | RKKRRQRRR |
| transduction domain | |
| pep-1 | KETWWETWWTEW |

In one embodiment of the invention, the Targ part of the polyfunctionnal molecule TARG-(MLS)-TOX is the decanoic acid CH₃(CH₂)₈CO—.

In one embodiment of the invention, the TARG part of the polyfunctional molecule TARG-(MLS)-TOX is an antibody, a recombinant antibody, a recombinant antibody fragment or a ScFv (single chain fragment variable).

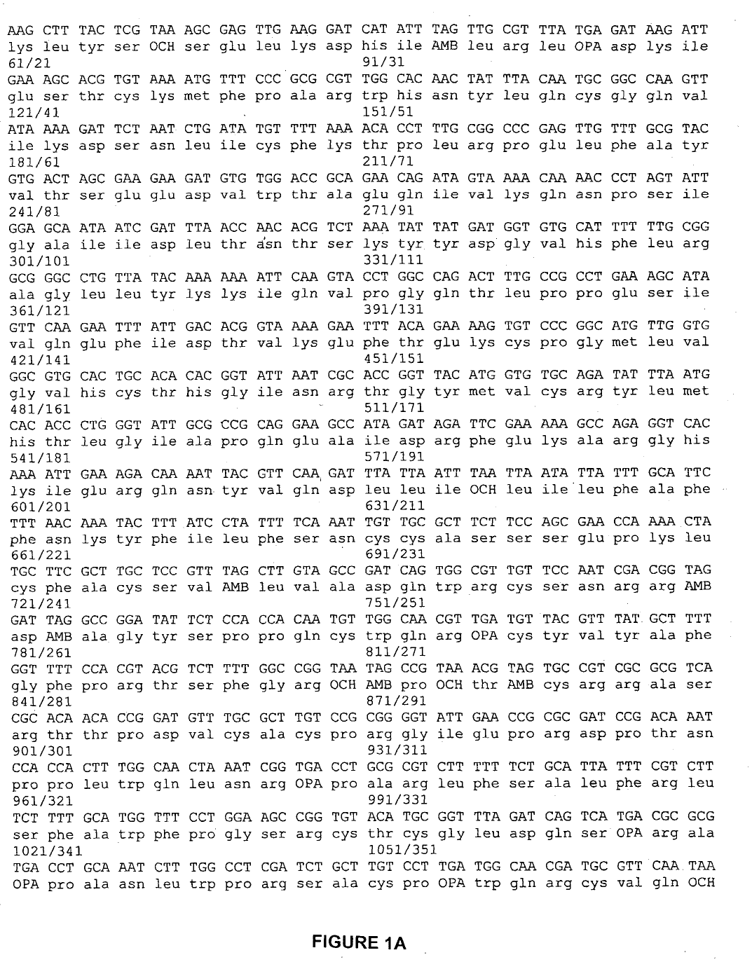

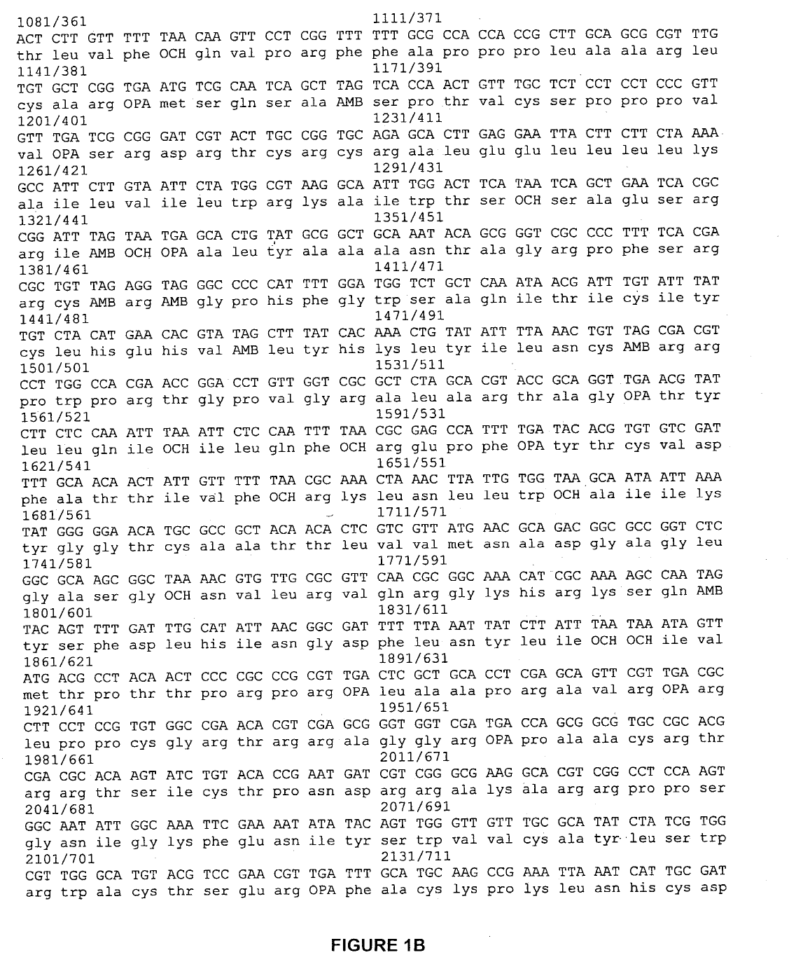

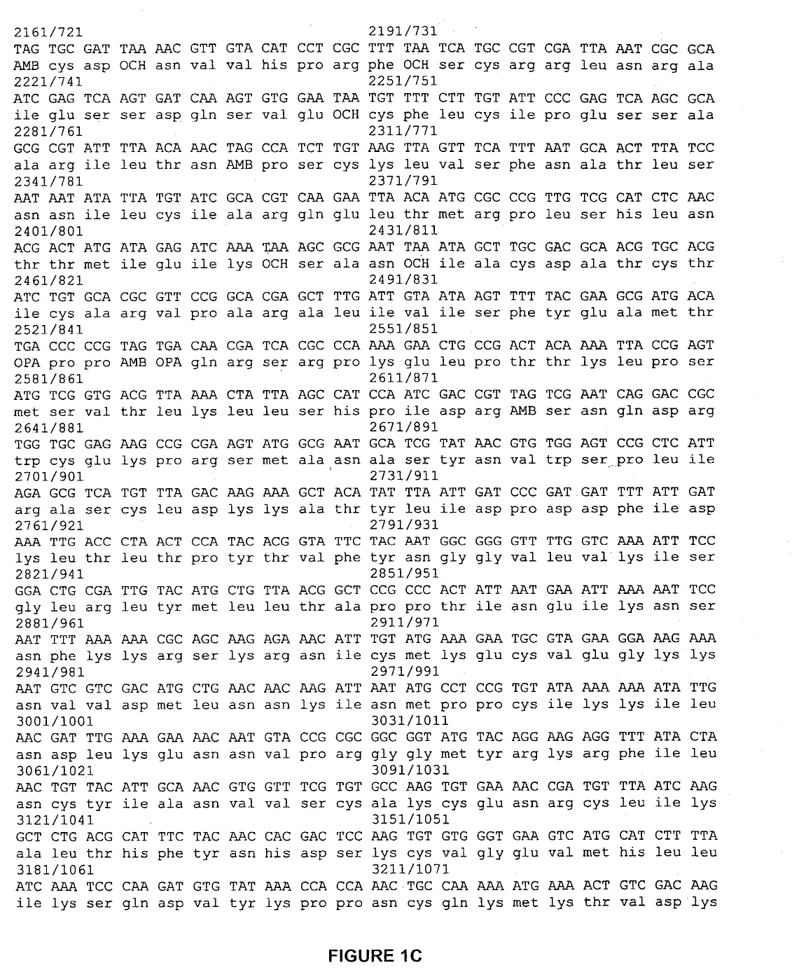

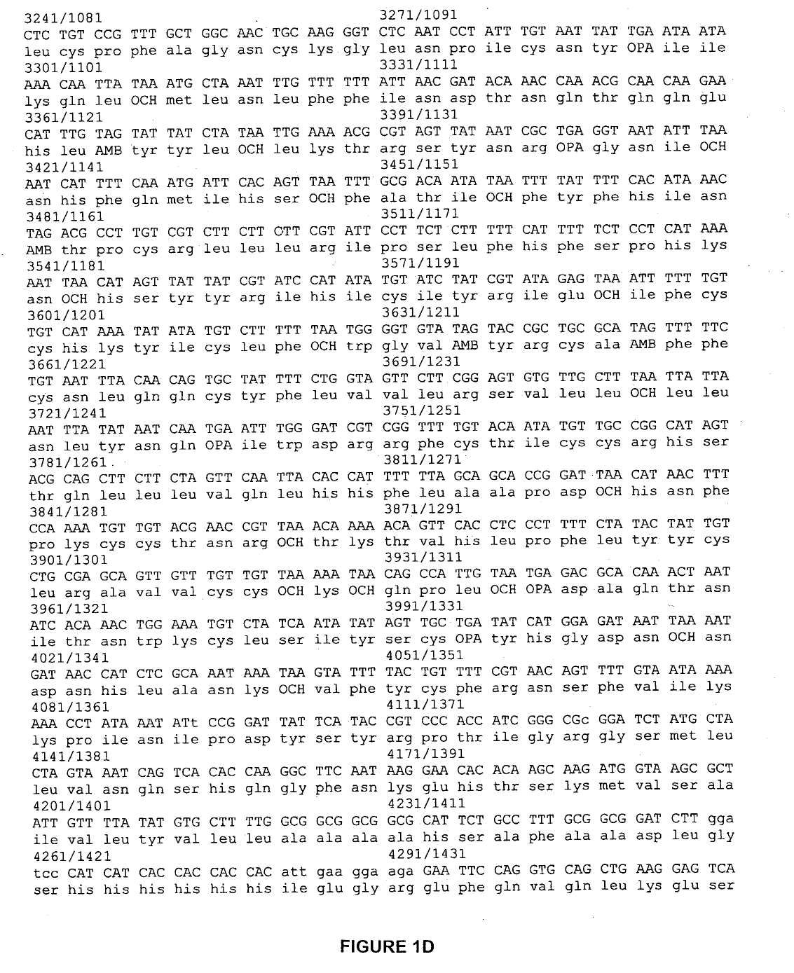

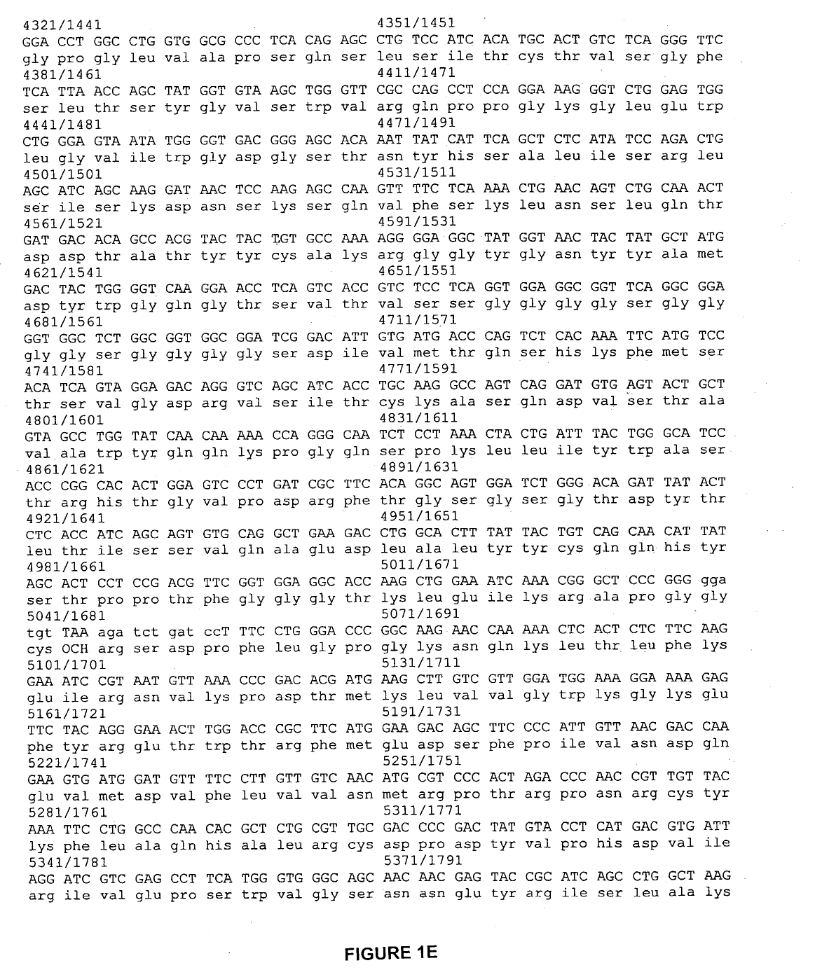

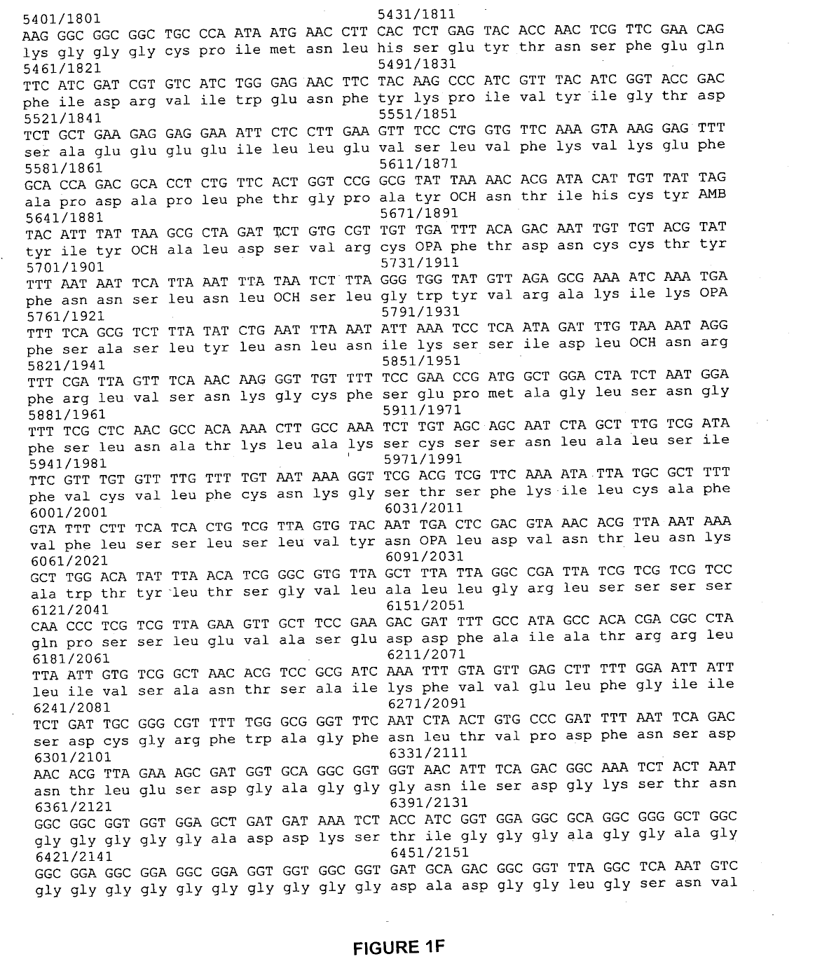

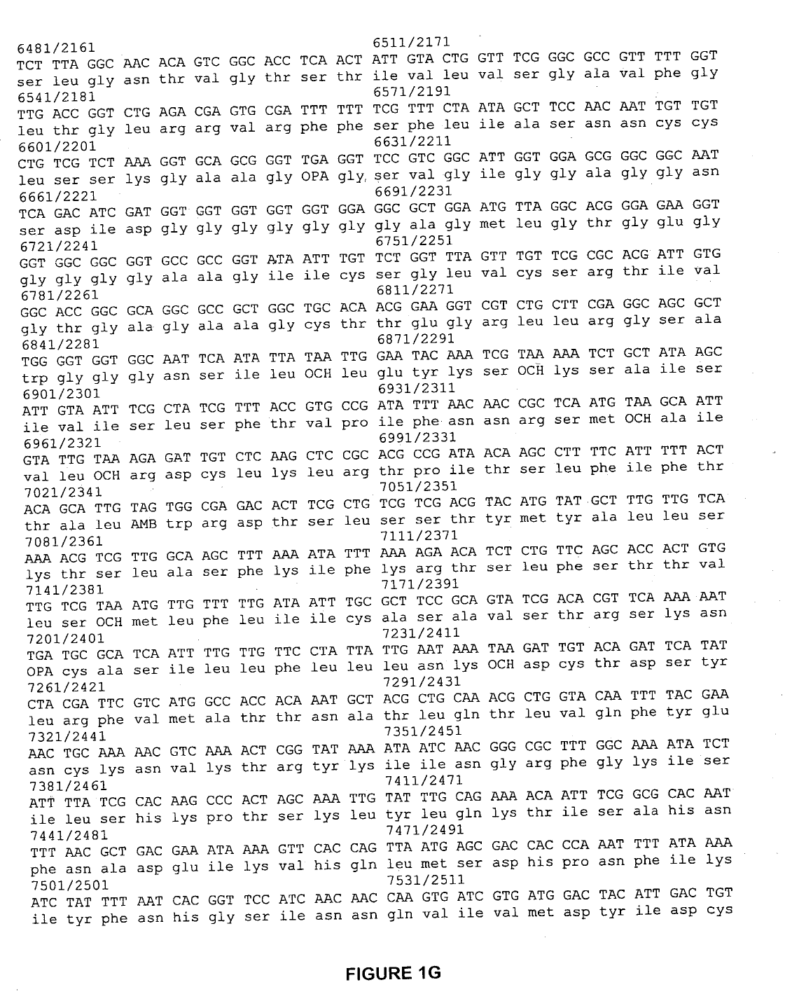

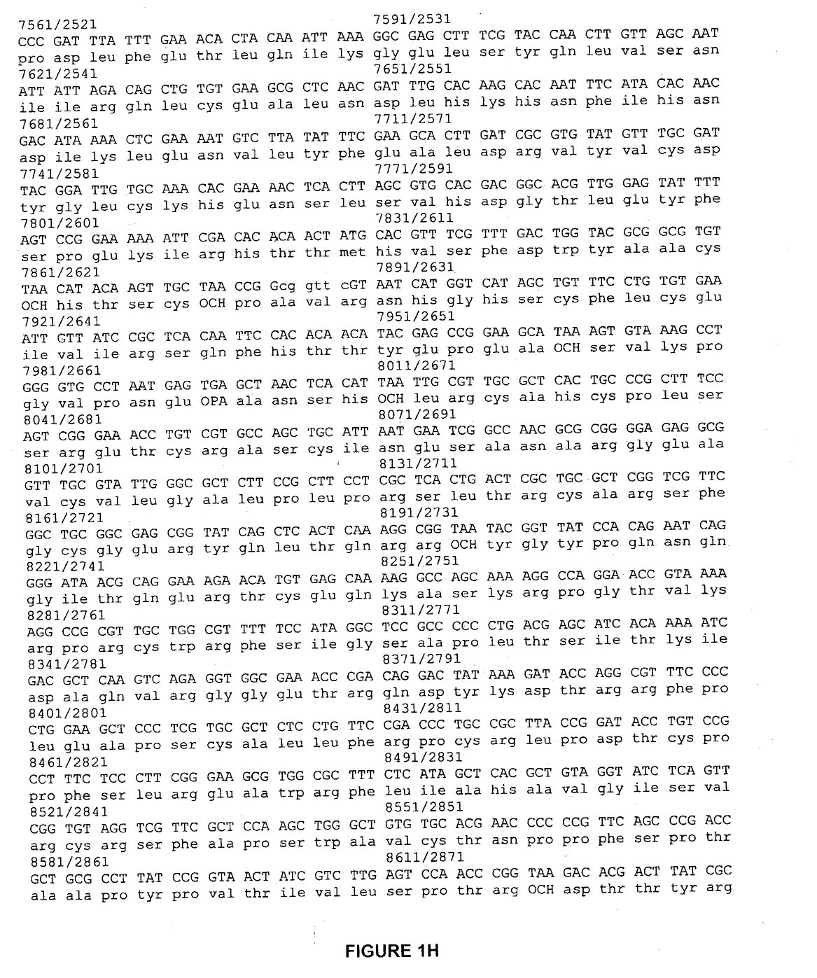

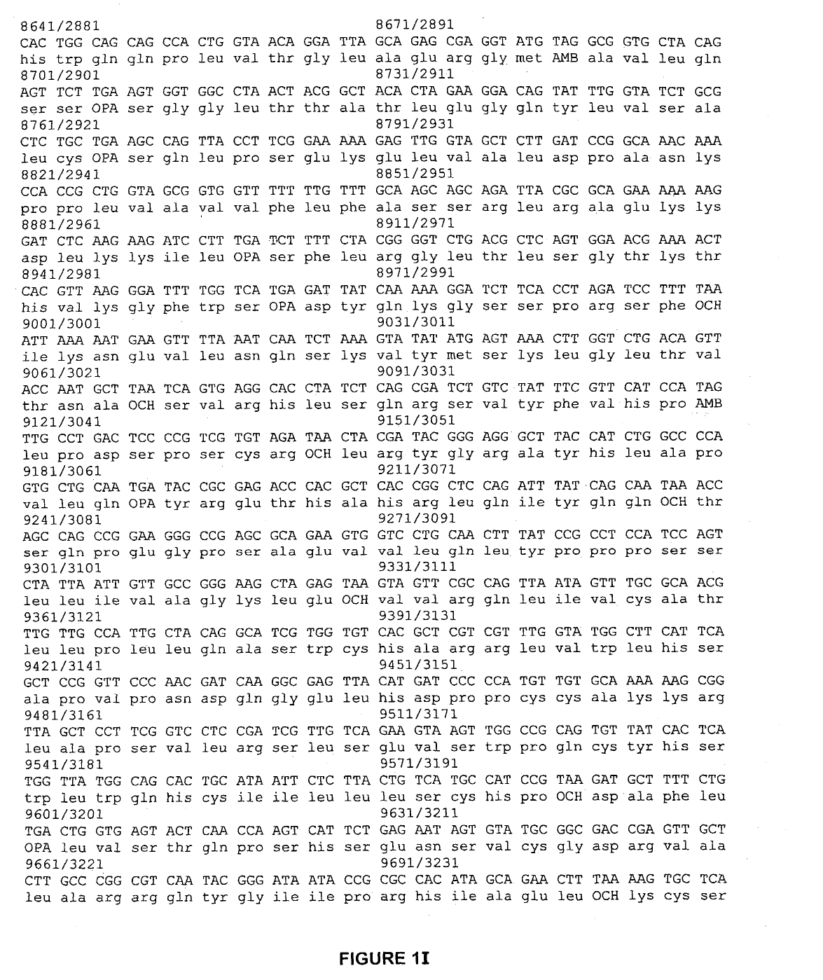

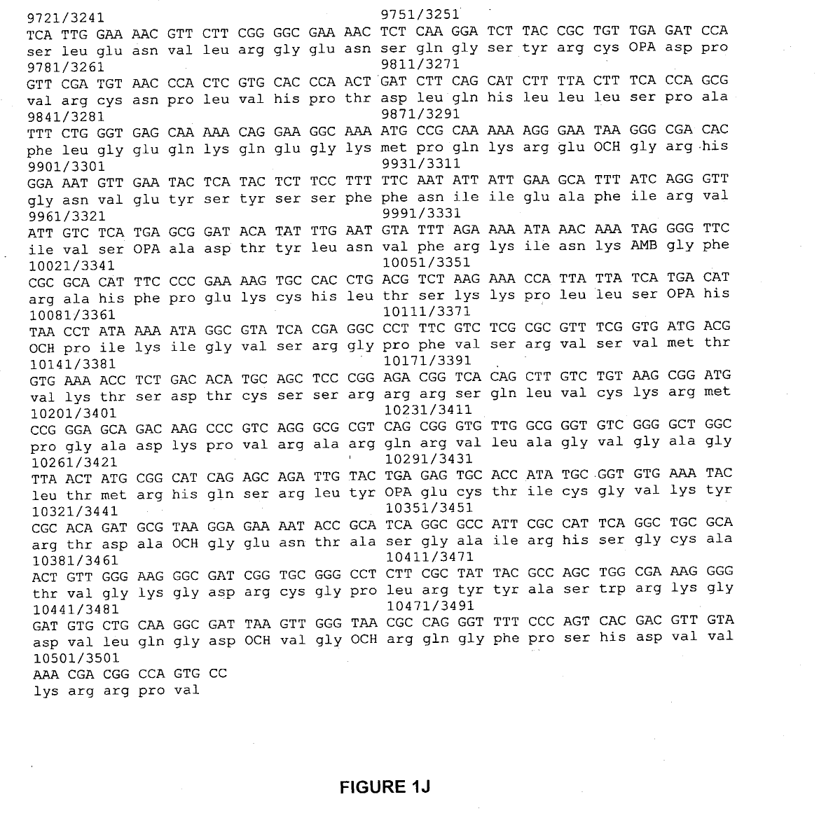

In one embodiment of the invention, the TARG part of the polyfunctional molecule TARG-(MLS)-TOX is encoded by the following vector pACgp67-ScFv461 (FIG. 1).

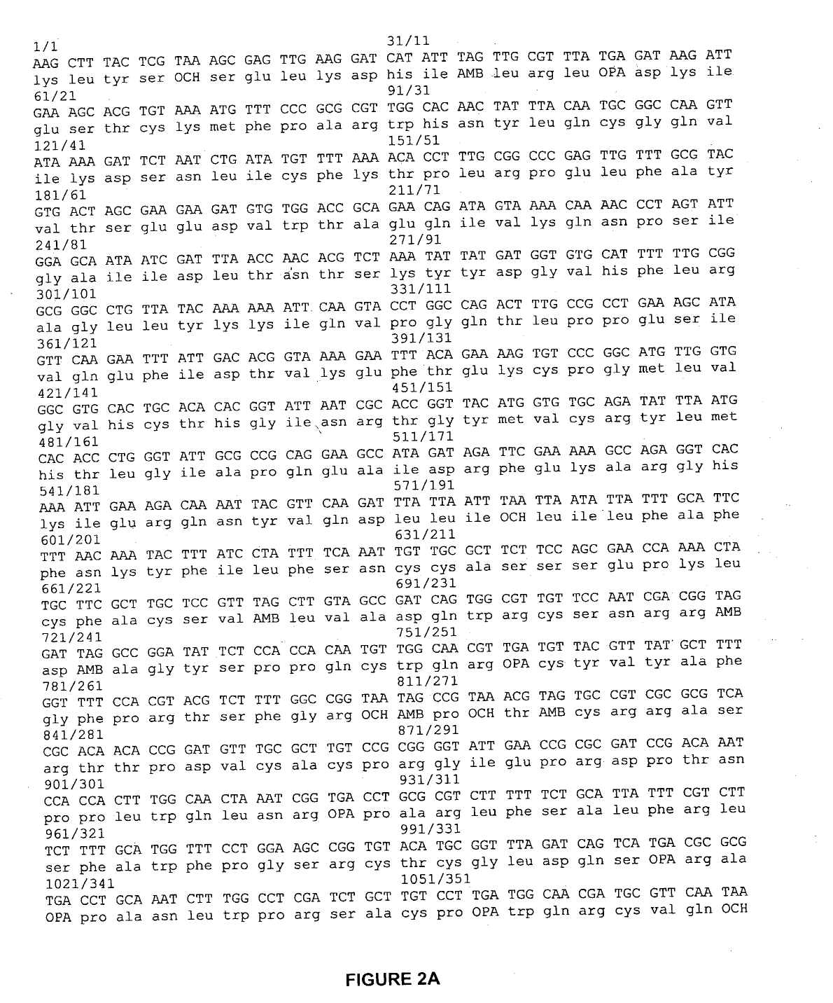

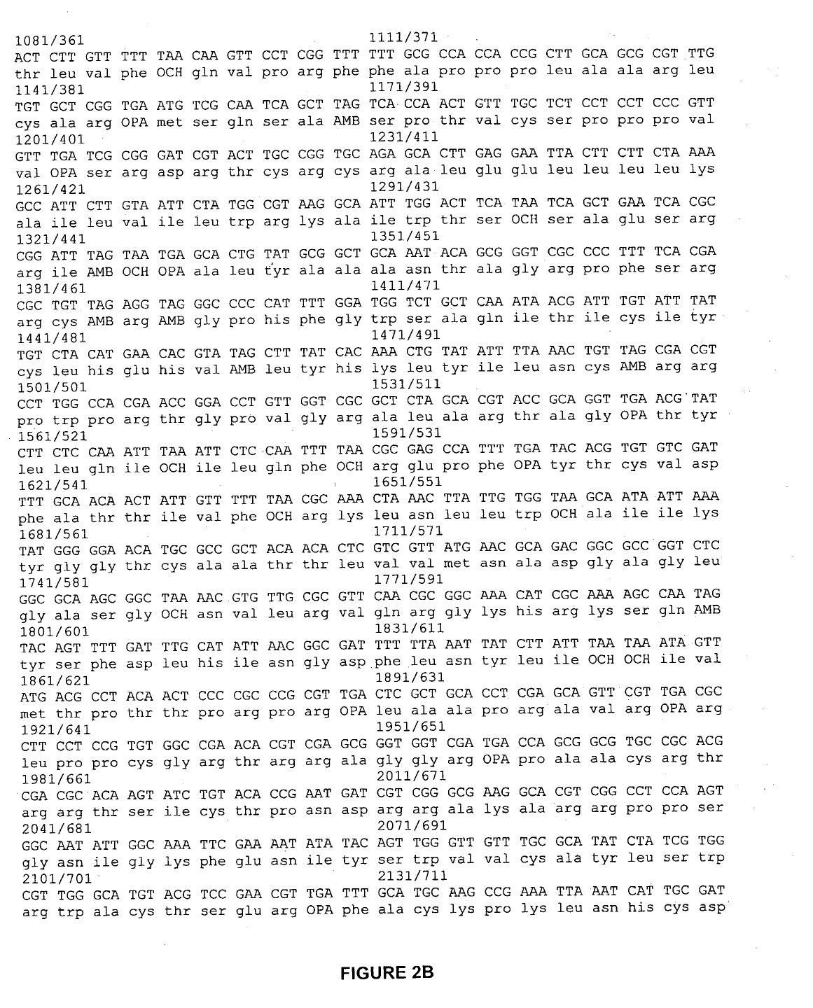

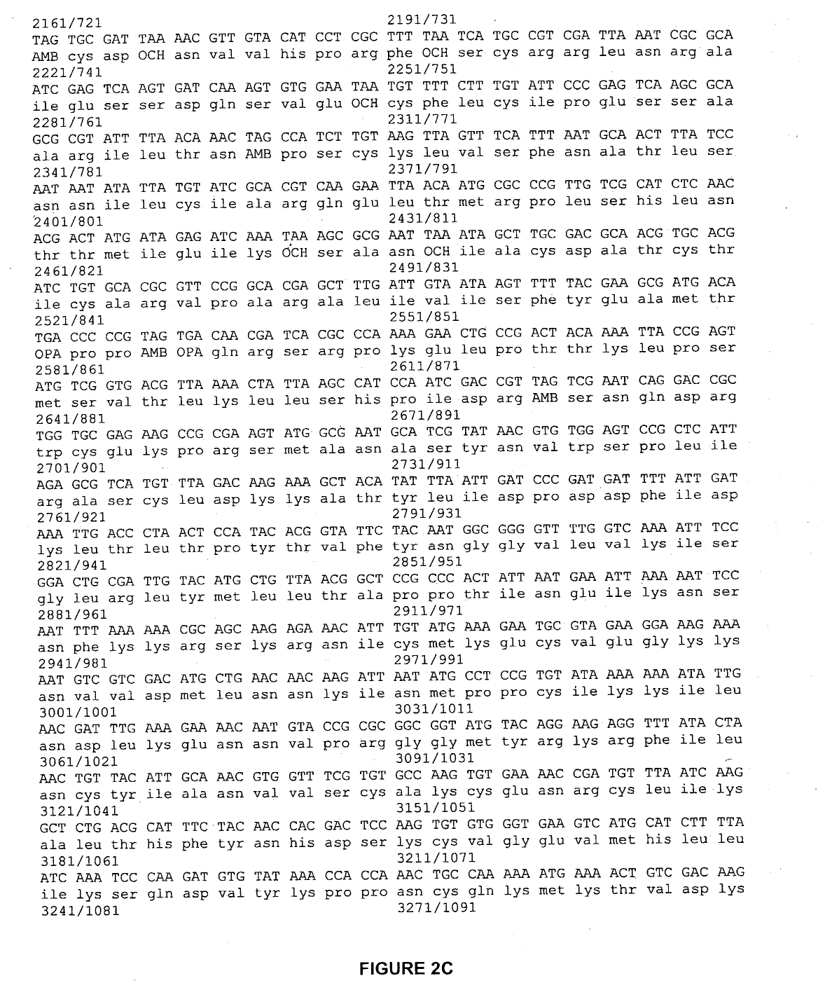

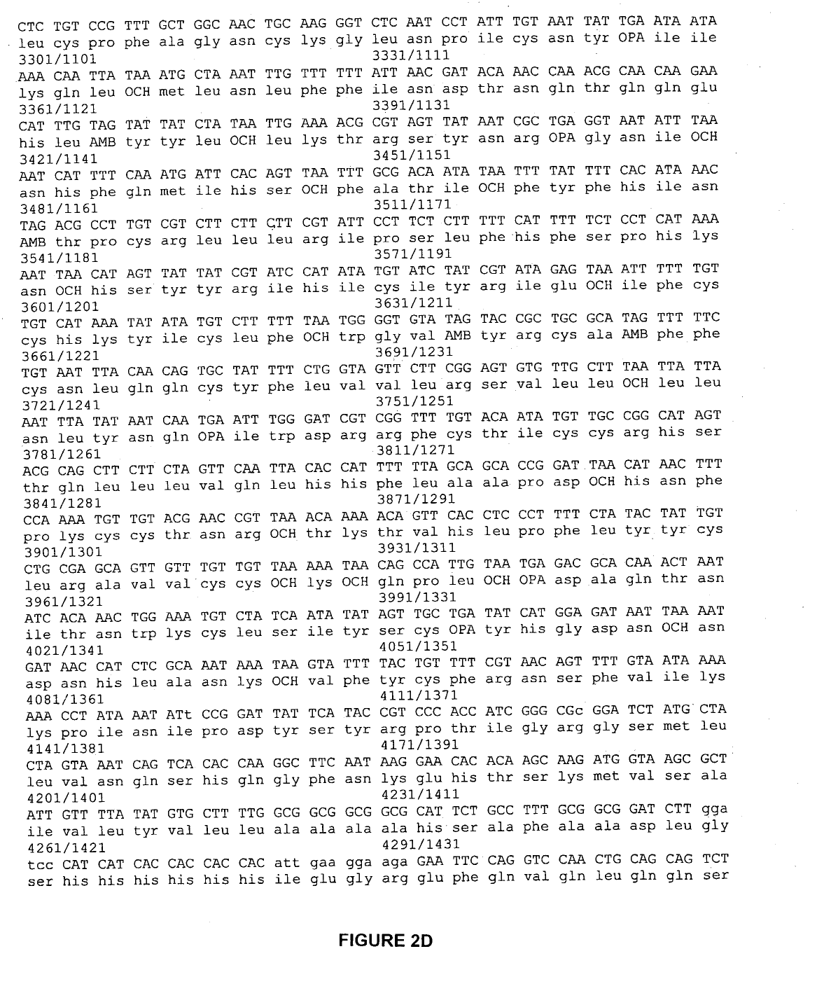

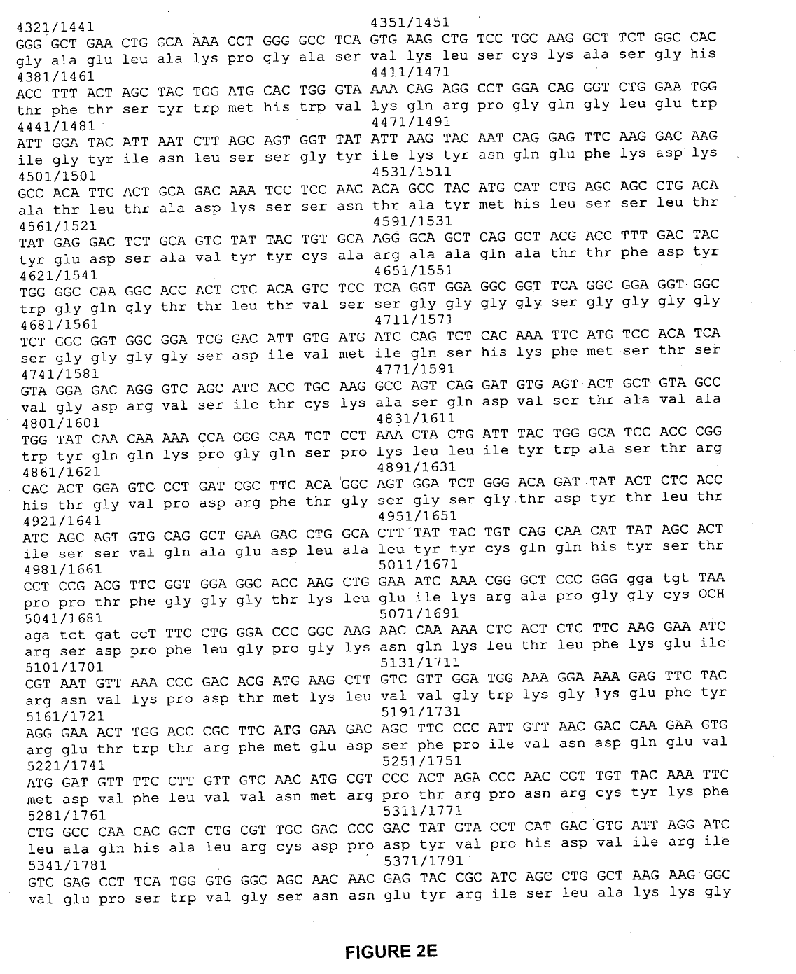

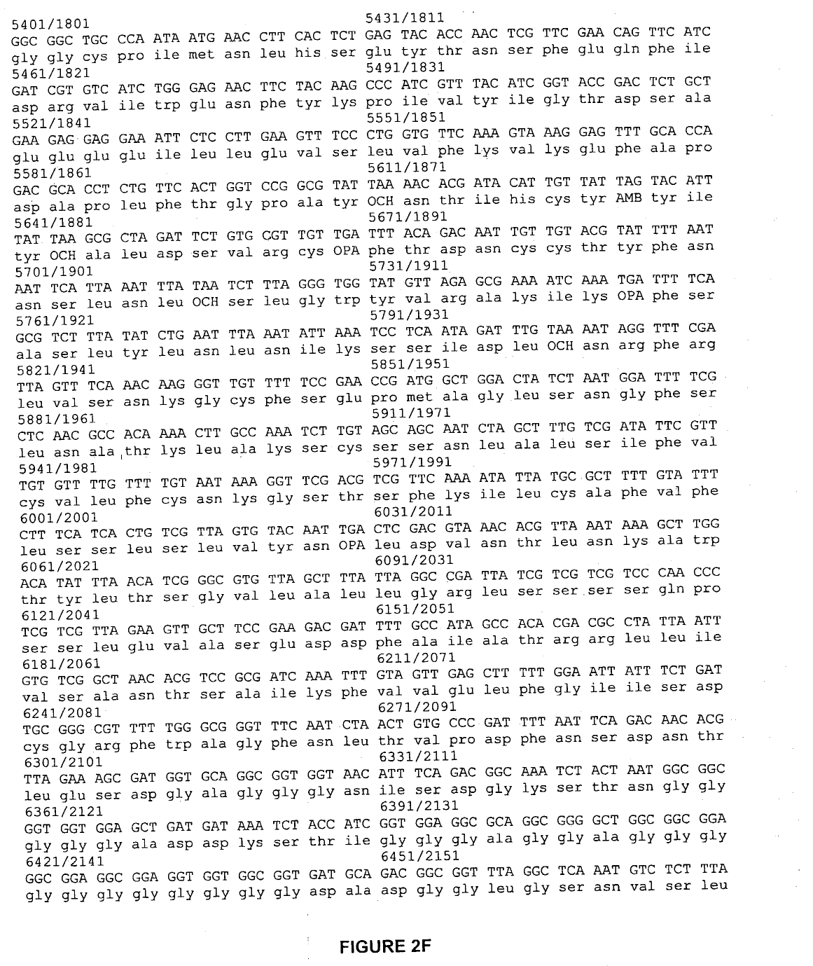

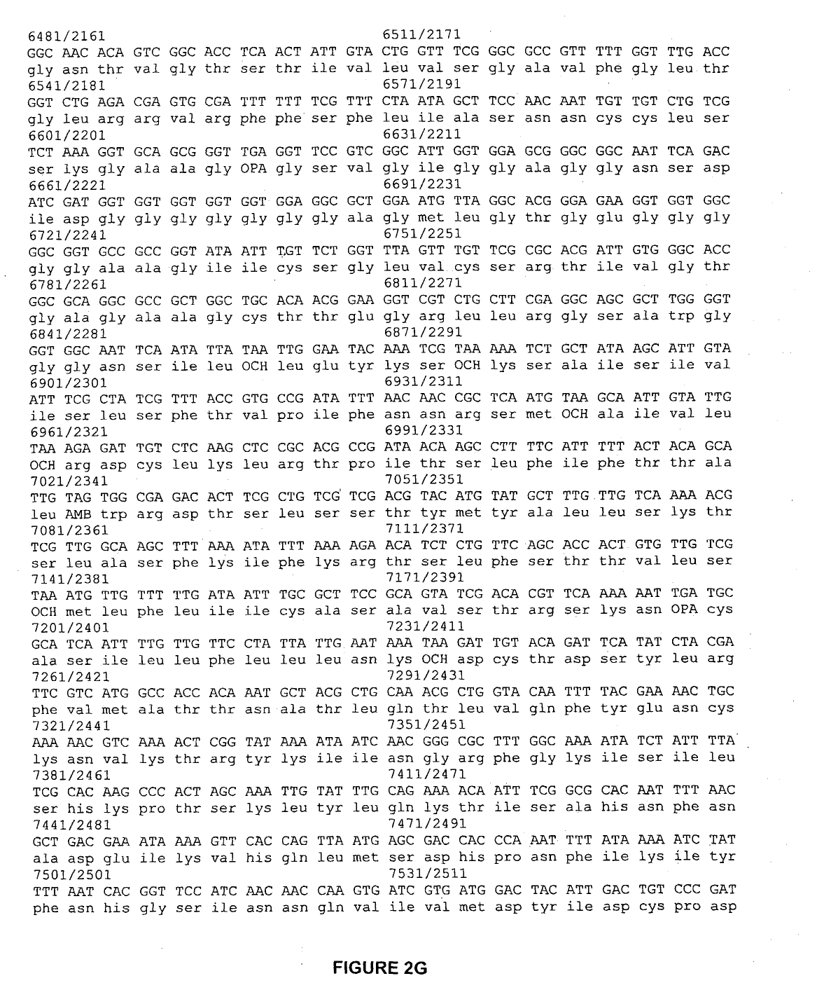

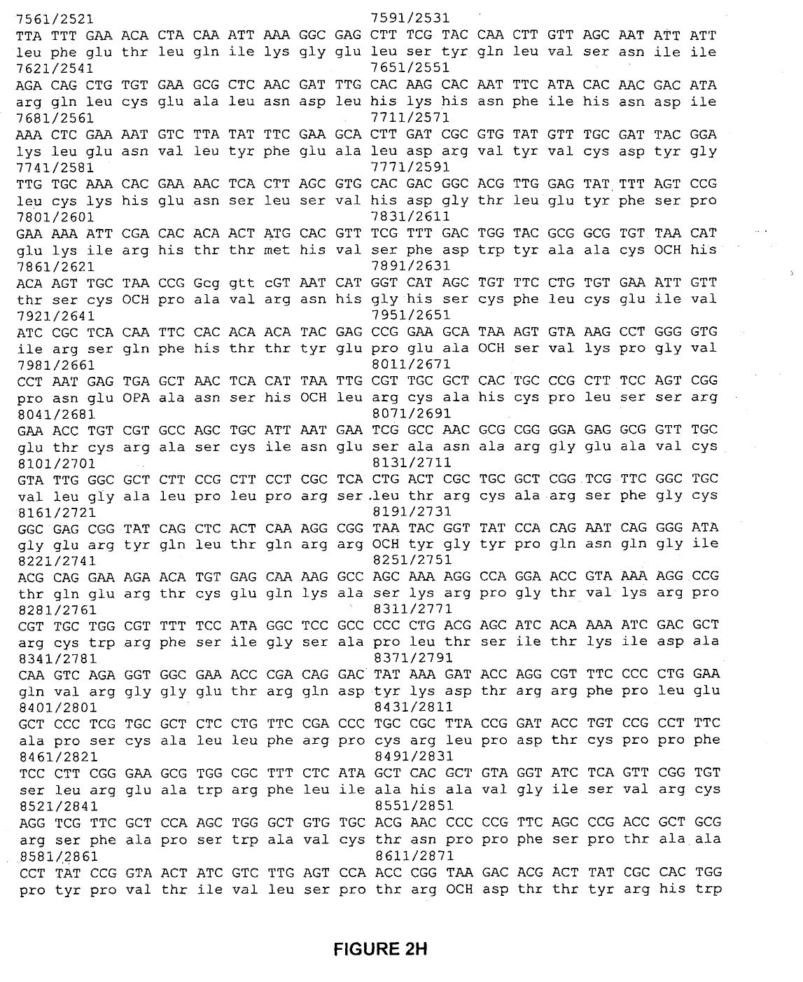

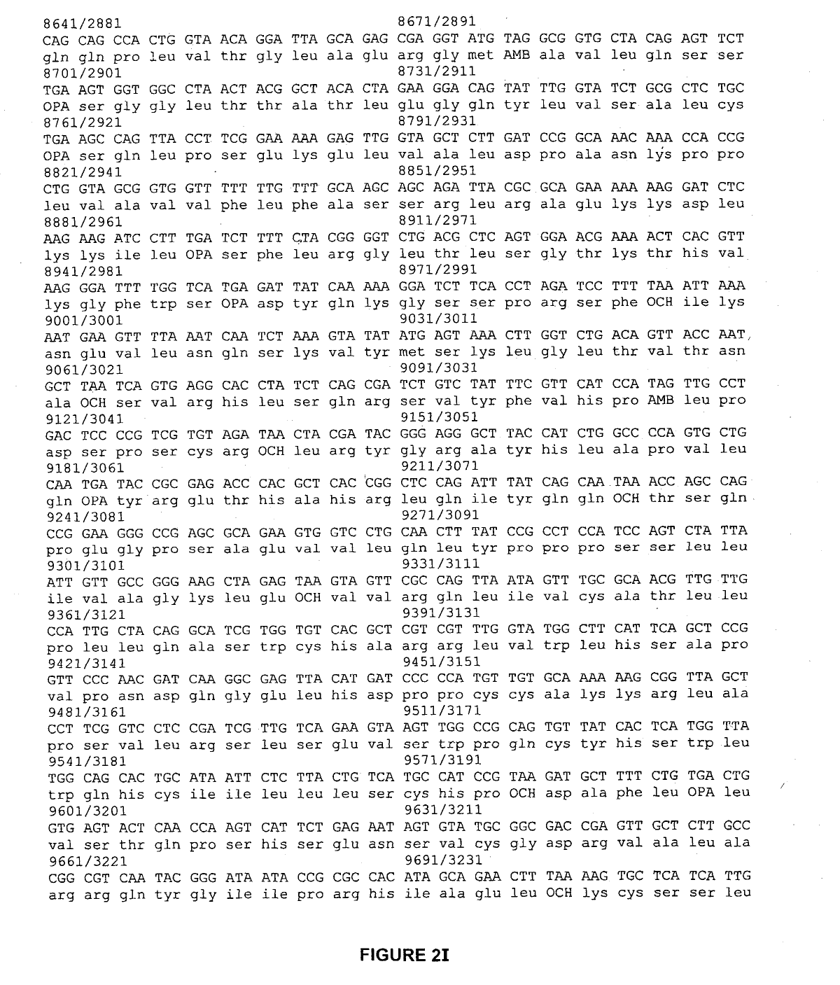

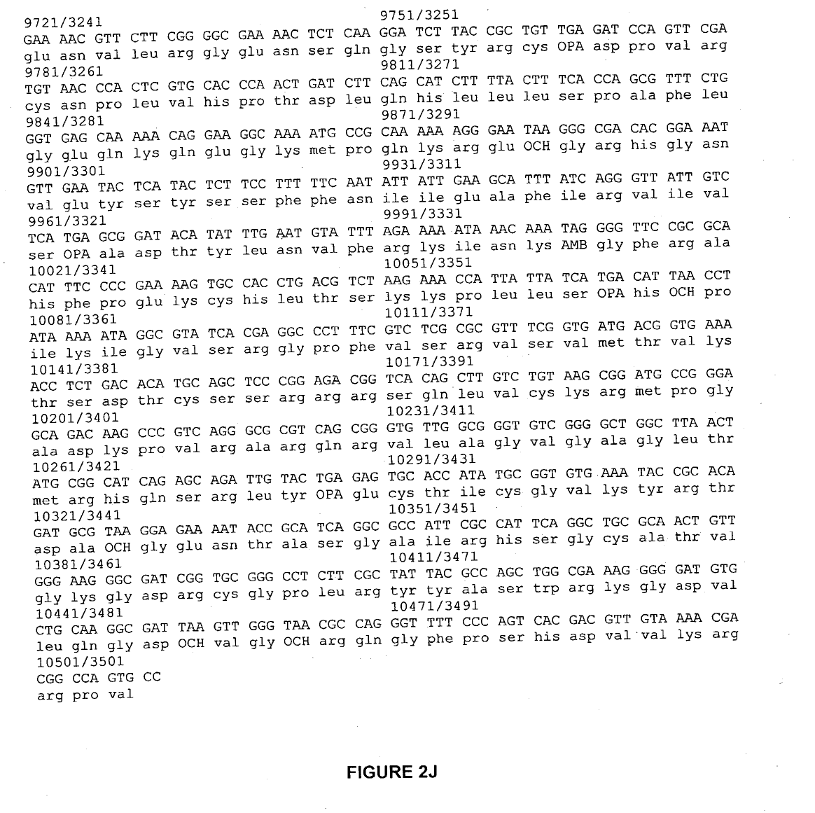

In one embodiment of the invention, the TARG part of the polyfunctional molecule TARG-(MLS)-TOX is encoded by the following vector pACgp67-ScFv350 (FIG. 2).

In one embodiment of the invention, the TARG part of the polyfunctional molecule TARG-(MLS)-TOX is a tumor homing peptide as defined by Ellerby et al in PCT/US00/01602.

In one embodiment of the invention, the TARG part of the polyfunctional molecule TARG-(MLS)-TOX/SAVE is a brain or kidney homing peptide as defined by Pasqualini R, Ruoslahti (in Nature Mar. 28, 1996;380(6572):364-6. Organ targeting in vivo using phage display peptide libraries).

In one embodiment of the invention, pTox is the Vpr peptide of HIV-1 or a fragment thereof. Protein R (Vpr) of human immunodeficiency virus type 1 (HIV-1) is a virion-associated viral gene product with an average length of 96 amino acids, and a molecular weight of approximately 15 kD. Vpr is a highly conserved viral protein among HIV, simian immunodeficiency viruses (SIV). See Yuqi Zhao and Robert T. Elder, “Yeast Perspectives on HIV-1 VPR,” Frontiers in Bioscience 5, d905-916, Dec. 1, 2000.

Vpr has been characterized as an oligomer, and is thought to be divided into three domains on the basis of its structural features: an amino-terminal, negatively charged region that is predicted to form an amphipathic α helix (amino acids 17 to 34); a central hydrophobic domain (amino acids 35 to 75); and a carboxy-terminal, positively charged domain (amino acids 80 to 96). Mutational analysis of Vpr suggests that the nuclear import, virion incorporation, and cell cycle arrest of Vpr are mediated by the distinct functional domains. A structural motif within an amino-terminal helix appears to be important for packaging of Vpr into virions and for maintaining the stability of the protein. A central hydrophobic region, especially the leucine- isoleucine (LR) domain, is reported to be involved in the nuclear localization of Vpr. The cell cycle arrest function of Vpr was found to be largely located within a carboxy-terminal, positively charged region. See Tomoyuki Yamaguchi, Nobumoto Watanabe, Hiromitsu Nakauchi, and Atsushi Koito, “Human Immunodeficiency virus type 1 Vpr Modifies Cell Proliferation via Multiple Pathways,” Microbiol, Immunol., 43(5), 437-447, 1999.

The amino acid sequence of human immunodeficiency virus type 1 viral protein R (Vpr) is shown below:

MEQAPEDQGPQREPYNEWTLELLEELKSEAVRHFPRIWLHNLGQHIYE TYGDTWAGVEAIIRILQQLLFIHFRIGCRHSRIGVTRQRRARNGASRS.

Vpr and peptides containing conserved H(F/S)RIG repeat motifs can rapidly penetrate human CD4 cells, and cause mitochondrial dysfunction and death by apoptosis. More particularly, recombinant Vpr and C-terminal peptides of Vpr containing the conserved sequence HFRIGCRHSRIG can cause permeabilization of CD4⁺ T lymphocytes, a dramatic reduction of mitochondrial membrane potential, and finally cell death. Vpr and Vpr peptides containing the conserved sequence rapidly penetrate cells, co-localize with the DNA, and cause increased granularity and formation of dense apoptotic bodies. Vpr treated cells undergo apoptosis, and this was confirmed by demonstration of DNA fragmentation. See C. Arunagiri, I. Macreadie, D. Hewish and A. Azad, “A C-terminal domain of HIV-1 accessory protein Vpr is involved in penetration, mitochondrial dysfunction and apoptosis of human CD4⁺ lymphocytes,” Apoptosis 1997; 2: 69-76.

Using a yeast model system, it has been confirmed that there is a cytocidal activity associated with the C-terminal portion of Vpr, particularly the sequence HFRIGCRHSRIG. Vpr and portions of Vpr containing the sequence HFRIGCRHSRIG can kill a range of mammalian cells including human lymphocytes. See I. G. Macreadie, A, Kirkpatrick, P. M. Strike, and A. A. Azad, “Cytocidal Activities of HIV-1 VPR and Sac1p peptides Bioassayed in Yeast,” Protein and Peptide Letters, Vol. 4, No. 3, pp. 181-186, 1997.

The C-terminal moiety (Vpr52-96), within an α-helical motif of 12 amino acids (Vpr71-82), contain several critical arginine (R) residues (R73, R77, R80), which are strongly conserved among different pathogenic HIV-1 isolates. L. G. Macreadie, et al., Proc. Natl. Acad. Sci. USA 92, 2770-2774 (1995). I. G. Macreadie, et al., FEBS Lett. 410, 145-149 (1997). E. Jacotot, et al., J. Exp. Med. 191, 33-45 (2000). Thus, the pro-apoptotic portion (pTox) of the chimeric polypeptide of the invention can contain, for example, the sequence HFRIGCRHSRIG (HIV-1 Vpr71-82), HFKIGCKHSKIG, Vpr 71-96, Vpr 52-96, or a pseudo peptidic variant such as D[HFRIGCRHSRIG].

Other variants of Vpr peptides can also be employed in this invention. Peptide fragments of Vpr encompassing a pair of H(F/S)RIG sequence motifs (residues 71-75 and 78-82 of HIV-1 Vpr) have been shown cause cell membrane permeabilization and death in yeast and mammalian cells. Peptide Vpr⁵⁹⁻⁸⁶ (residues 59-86 of Vpr) forms an α-helix encompassing residues 60-77, with a kink in the vicinity of residue 62. It has been shown that the first of the repeated sequence motifs (HFRIG) participates in a well-defined α-helical domain, whereas the second (HSRIG) lay outside the helical domain and forms a reverse turn followed by a less ordered region. On the other hand, peptides Vpr⁷¹⁻⁸² and Vpr⁷¹⁻⁹⁶, in which the sequence motifs are located at the N-terminus, were largely unstructured under similar conditions, as judged by their C²H chemical shifts. Thus, it has been shown that the HFRIG and HSRIG motifs adopt α-helical and turn structures, respectively, when preceded by a helical structure, but are largely unstructured in isolation. There are implications of these findings for interpretation of the structure-function relationships of synthetic peptides containing these motifs. For example, since the HFRIG and HSRIG sequence motifs adopt helical and turn structures, respectively, when preceded by a helical structure, as in full-length Vpr, but are largely unstructured in isolation, 7-8 residues, sufficient to support at least 1-2 turns of helix, should be included at the N-terminus of Vpr when used as the pTox component of the chimeric polypeptides of the invention to ensure that they are able to adopt the same structure as in the full-length protein. See Shenggen Yao, Allan M. Torres, Ahmed A. Azad, Ian G. Macreadie and Raymond S. Norton, “Solution Structure of Peptides from HIV-1 Vpr Protein that Cause Membrane Permeabilization and Growth Arrest,” J. Peptide Sci. 4: 426-435 (1998). While the Vpr gene codes for a protein of 96-amino-acids, variations have been observed, e.g., Vprs from HIV-1_{HXB2} have 97 and 90-amino-acid residues, respectively. It will be understood that these variants can also be employed in this invention.

For the most effective toxicity, HFRIGCRHSRIG should be surrounded on each side by about eight amino acids from the native sequence. Vpr polypeptides and peptides of greater than 9 amino acids that inhibit or augment Vpr binding, mitochondrial membrane permeabilization, or apoptosis can also be employed in the invention, as well as peptides that are at least 10-20, 20-30, 30-50, 50-100, and 100-365 amino acids in size. DNA fragments encoding these polypeptides and peptides are encompassed by the invention. Flanking residues should not disrupt the helical structures described above.

The Vpr variants and other viral apoptotic peptides can be assessed for their ability to mediate apoptosis, and thus their suitability for use as pTox in the invention. It is understood that many techniques could be used to assess binding of Vpr or another viral apoptotic peptide to ANT, and that these embodiments in no way limit the scope of the invention. For example, in one embodiment, surface plasmon resonance is used to assess binding of Vpr or another viral apoptotic peptide to ANT. In another embodiment, electrophysiology is used to assess binding of Vpr or another viral apoptotic peptide to ANT. In another embodiment, purified mitochondria are used to assess binding of Vpr or another viral apoptotic peptide to ANT. In another embodiment, synthetic proteoliposomes are used to assess binding of Vpr or another viral apoptotic peptide to ANT. In another embodiment, microinjection of live cells is used to assess binding of Vpr or another viral apoptotic peptide to ANT. These techniques are described in U.S. Provisional Application No. 60/231,539.

In another embodiment, the yeast two-hybrid system developed at SUNY (described in U.S. Pat. No. 5,282,173 to Fields et al.; J. Luban and S. Goff., Curr Opin. Biotechnol. 6:59-64, 1995; R. Brachmann and J. Boeke, Curr Opin. Biotechnol. 8:561-568, 1997; R. Brent and R. Finley, Ann. Rev. Genet. 31:663-704, 1997; P. Bartel and S. Fields, Methods Enzymol. 254:241-263, 1995) can be used to screen for Vpr-ANT interaction as follows. Vpr, or portions thereof, or another viral apoptotic peptide, responsible for interaction, can be fused to the Gal4 DNA binding domain and introduced, together with an ANT molecule fused to the GAL 4 transcriptional activation domain, into a strain that depends on GAl4 activity for growth on plates lacking histidine. Interaction of the Vpr polypeptide or another viral apoptotic peptide with an ANT molecule allows growth of the yeast containing both molecules and allows screening for the molecules that inhibit or alter this interaction (i.e., by inhibiting or augmenting growth). In an alternative embodiment, a detectable marker (e.g. β-galactosidase) can be used to measure binding in a yeast two-hybrid assay.

Alternatively, the binding properties of Vpr peptide fragments or another viral apoptotic peptide can be determined by analyzing the binding of Vpr peptide fragments or another viral apoptotic peptide to ANT-expressing cells by FACS analysis. This allows the characterization of the binding of the peptides, and the discrimination of relative abilities of the peptide to bind to ANT. In vitro binding assays with Vpr or another viral apoptotic peptide can similarly be used to characterize ANT binding activity.

In another specific embodiment, a cytotoxic conjugate of the invention includes an adenine nucleotide translocation (ANT)-derived pro-apoptotic peptide. The pro-apoptotic portion (pTox) of the conjugate can contain, for example, the sequence DKRTQFWRYFPGN (hANT₂104-116[A114P]) or a pseudo-peptidic variant such as [DKRTQFWRYFPGN].

In another specific embodiment, a cytoprotective conjugate of the invention includes ANT-derived anti-apoptotic peptides. The anti-apoptotic portion (pSave) of the conjugate can contain, for example, the sequence DKRTQFWRYFAGN (hANT₂104-116), the sequence LASGGAAGATSLCFVYPL (ANT 117-134) or a pseudo-peptidic variant such as D[DKRTQFWRYFPGN].

The pTarg component of the chimeric polypeptide of the invention can be an antibody or an antibody fragment. The antibody or antibody fragment can be all or part of a polyclonal or monoclonal antibody. The term “antibodies” is meant to include polyclonal antibodies, monoclonal antibodies, fragments thereof, as well as any recombinantly produced binding partners. Antibodies are defined to be specifically binding if they bind with a Kₐ or greater than or equal to about 10⁷ M⁻¹. Affinities of binding partners or antibodies can be readily determined using conventional techniques, for example those described by Scatchard et al., Ann. N.Y. Acad. Sci., 51:660 (1949).

As used herein, the term “antibody fragment” includes the following:

| | |
|---|---|
| Fc | A constant region dimer lacking C_{H}1 |
| Fab | A light chain dimerized to V_{H}—C_{H}1 resulting from |
| | papain cleavage; this is monomeric since papain cuts |
| | above the hinge cystines |
| F(ab)′₂ | A dimer of Fab′ resulting from pepsin cleavage below |
| | the hinge disulfides; this is bivalent and can precipitate |
| | antigen |
| Fab′ | A monomer resulting from mild reduction of F(ab)′₂: |
| | an Fab with part of the hinge |
| Fd | The heavy chain portion of Fab (V_{H}—C_{H}1) obtained |
| | following reductive denaturation of Fab |
| Fv | The variable part of Fab: a V_{H}—V_{L} dimer |
| Fb | The constant part of Fab: a C_{H} 1-C_{L} dimer |
| pFc′ | A C_{H}3 dimer |

Fragments of monoclonal antibodies are of particular interest as small antigen targeting molecules. Antibody fragments are also useful for the assembly of the chimeric polypeptides of the invention designed to carry other pTox agents, such as a therapeutic conjugate. For in vivo applications, fragments of antibodies are of interest due to their altered pharmacokinetic behavior, which is useful for cancer therapy with cytotoxic agents, and for their rapid penetration into body tissues, which offer advantages for therapy techniques.

An antibody fragment of particular interest for use in the invention is a minimal Fv fragment with antigen-binding activity. The two chains of the Fv fragment are less stably associated than the Fd and light chain of the Fab fragment with no covalent bond and less non-covalent interaction, but nevertheless functional Fv fragments have been expressed for a number of different antibodies. Two strategies can be employed to stabilize the Fv fragments used in the invention: firstly, mutating a selected residue on each of the V_{H} and V_{L} chains to a cysteine to allow formation of a disulphide bond between the two domains; and secondly, the introduction of a peptide linker between the C-terminus of one domain and the N-terminus of the other, such that the Fv is produced as a single polypeptide chain known as a single-chain Fv.

Thus, single-chain Fvs (ScFvs), recombinant V_{L} and V_{H} fragments covalently tethered together by a polypeptide link and forming one polypeptide chain, are useful in this invention. For expression of Fv genes, several systems can be effectively used, including myeloma cells, insect, yeast, and Escherichia coli cells. Expression in E. coli has been a frequently used production method, with both intracellular expression and secretion enabling high yields of ScFv to be made.

The production of ScFv molecules requires the identification of a suitable peptide linker to span the 35-40 Å distance between the C-terminus of one domain and the N-terminus of the other and allow correct folding and assembly of the Fv structure. Several different types of linkers have been used and shown to result in functional ScFv. Polypeptides with the average length of 3-18 amino acids are usually used as links. They can be rich in serine and/or glycine residues, which introduce flexibility, or in charged glutamic acid and/or lysine residues, which improve solubility. Linkers can be selected from searching existing protein structures for protein fragments of the appropriate length and conformation, or by designing them de novo based on simple, flexible structures, such as the 15 amino acid sequence (Gly₄Ser)₃.

Active single-chain Fv molecules in both of the two possible orientations, V_{H}-linker-V_{L} or V_{L}-linker-V_{H} are useful in the invention; however, for some antibodies one particular orientation may be preferable as a free N-terminus of one domain, or C-terminus of the other, may be required to retain the native conformation and thus full antigen binding.

The ScFv may be susceptible to aggregation, with dimers, trimers, and multimers formed. The potential of forming dimers or other multimers with very short linkers, or no linker at all, can be exploited to produce stable pTarg structures. Such an approach can also be used to create pTarg molecules with two different binding specificities by fusing the V_{H} of an antibody of one specificity to the V_{L} of another and vice versa.

Fv's stabilized by disulphide linkages can also be employed as the pTarg component of the chimeric polypeptide of the invention. The introduction of a disulphide bond between the V_{H} and V_{L} domains to form a disulphide-linked Fv requires the identification of residues in close proximity on each chain, which are unlikely to affect directly the conformation of the binding site when mutated to cysteine, and will be capable of forming a disulphide bond without introducing strain into the structure of the Fv. Sites have been identified in both CDR regions and framework regions, which appear to result in the formation of such disulphide bonds and allow the production of stabilized Fv fragments which retain antigen-binding characteristics.

Due to small size, rapid clearance in vivo, stability, and easy engineering, ScFvs employed in this invention have various applications in the treatment of diseases, particularly of cancer. ScFvs can exhibit the same affinity and specificity for antigen as monoclonal antibodies. Dozens of ScFvs with different specificities have been constructed. They are useful for genetic fusion to the potent toxins (pTox). If the monovalency of ScFv is a disadvantage, constructs with di- or multivalency with increased combining efficiency can be employed.

In a preferred embodiment of the invention, the targeting part (pTarg) of the cytotoxic conjugate is a recombinant portion (ScFv) of a tumor specific antibody, such as the ScFv versions of the M350 and V461 monoclonal antibodies. The hybridoma has been deposited at the CNCM on Jan. 24, 2001, under the Accession Number I-2617.

The pTarg component of the chimeric polypeptide of the invention is preferably a monoclonal antibody or a fragment thereof. Monoclonal antibodies to human cell antigens are preferred. Many tumor-associated antigens are now known and characterized, and antibodies to these allow targeting to different tumor types. Useful tumor-associated antigens are absent on normal tissues and present at high levels on tumor cells, preferably homogeneously on all cells of the tumor. Antigen should also not be shed from the tumor into the blood.

Commonly used tumor-associated antigens and examples of antibodies raised against them are described in the following Table.

| | | Represent- |
|---|---|---|
| | | ative |
| Antigen | Tumor type | antibody |
| Turmor-associated glycoprotein 72 | Pancarcinoma | B72.3, CC49 |
| (TAG72), 72 kDa glycoprotein | | |
| Carcinoembryonic antigen (CEA), | Pancarcinoma | NP-4, A5B7 |
| 180 kDa blycoprotein | | |
| Polymorphic epithelial mucin | Ovarian, breast, lung | HMFG1 |
| (PEM), >100 kDa glycoprotein | | |
| Epithelial membrane antigen | Colorectal (and other | 17-1A |
| (EMA), 40 kDa glycoprotein | epithelial tumors) | |
| epidermal growth factor receptor | Breast, lung | 425 |
| (EGFR), 175 kDa glycoprotein | | |
| p185^{HER2}/c-erb-B2 | Breast, lung | 4D5 |
| (185 kDa glycoprotein) | | |
| Prostate-specific membrane antigen | Prostrate | 7E11-C5.3 |
| (PSMA), 100 kDa glycoprotein | | |
| CD33 67 kDa glycoprotein | Myeloid leukemia | P67.6,M195 |
| CD 20 35 kDa glycoprotein | Lymphoma | C2B8 |
| GD2 ganglioside | Melanoma, | 14-18 |
| | neuroblastoma | |

An important consideration is the absolute amount of antibody localized to the tumor site. Therefore, the ideal molecule would localize to the tumor in large amounts, delivering a high dose of pTox while clearing rapidly from the circulation and the rest of the body, minimizing non-specific toxicity. Intact antibodies typically circulate for a long period of time and accumulate high levels of activity at the tumor site, whereas antibody fragments clear more rapidly, sparing the dose to normal tissues.

The antibody fragments can also be prepared by phage-display technology. Phage display is a selection technique, according to which an antibody fragment (ScFv) is expressed on the surface of the filamentous phage fd. For this, the coding sequence of the antibody variable genes is fused with the gene that encoded the minor coat phage protein III (g3p) located at the end of the phage particle. The fused antibody fragments are displayed on the virion surface and particles with the fragments can be selected by adsorption on insolubilized antigen (panning). The selected particles are used after elution to reinfect bacterial cells. The repeated rounds of adsorbtion and infection lead to enrichment. Bacterial proteases can cleave the bond between the g3p protein and antibody fragments, which results in the production of soluble antibody fragments by infected bacterial cells. To release the soluble ScFvs, an excision of the g3p gene is made or an amber stop codon between the antibody gene and the g3p gene is engineered.

Immunoglobins and certain variants thereof are known and many have been prepared in recombinant cell culture. For example, see U.S. Pat. No. 4,745,055; EP 256,654; Faulkner et al., Nature 298:286 (1982); EP 120,694; EP 125,023; Morrison, J. Immun. 123:793 (1979); Köhler et al., P.N.A.S. USA 77:2197 (1980); Raso et al., Cancer Res. 41:2073 (1981); Morrison et al., Ann. Rev. Immunol. 2:239 (1984); Morrison, Science 229:1202 (1985); Morrison et al., P.N.A.S. USA 81:6851 (1984); EP 255,694; EP 266,663; and WO 88/03559. Reassorted immunoglobulin chains also are known. See for example U.S. Pat. No. 4,444,878; WO 88/03565; and EP 68,763 and references cited therein. DNA encoding immunoglobulin light or heavy chain constant regions is known or readily available from cDNA libraries or is synthesized. See for example, Adams et al., Biochemistry 19:2711-2719 (1980); Gough et al., Biochemistry 19:2702-2710 (1980); Dolby et al., P.N.A.S. USA, 77:6027-6031 (1980); Rice et al., P.N.A.S. USA 79:7862-7865 (1982); Falkner et al., Nature 298:286-288 (1982); and Morrison et al., Ann. Rev. Immunol. 2:239-256 (1984). These materials and techniques can be employed to synthesize the pTarg component of the chimeric polypeptide of the invention.

Polyclonal antibodies employed as the pTarg component of the chimeric polypeptide of the invention can be readily generated from a variety of sources, for example, horses, cows, goats, sheep, dogs, chickens, rabbits, mice, or rats, using procedures that are well known in the art. In general, purified cell surface proteins or glycoproteins or a peptide based on the amino acid sequence of cell surface proteins or glycoproteins that is appropriately conjugated is administered to the host animal typically through parenteral injection. The immunogenicity of cell surface proteins or glycoproteins can be enhanced through the use of an adjuvant, for example, Freund's complete or incomplete adjuvant. Following booster immunizations, small samples of serum are collected and tested for reactivity to cell surface proteins or glycoproteins. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies employed as the pTarg component can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKearn, and Bechtol (eds.), 1980. Briefly, the host animals, such as mice, are injected intraperitoneally at least once and preferably at least twice at about 3 week intervals with isolated and purified cell surface proteins or glycoproteins, conjugated cell surface proteins or glycoproteins, optionally in the presence of adjuvant. Mouse sera are then assayed by conventional dot blot technique or antibody capture (ABC) to determine which animal is best to fuse. Approximately two to three weeks later, the mice are given an intravenous boost of cell surface proteins or glycoproteins or conjugated cell surface proteins or glycoproteins. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out in plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as ¹²⁵I-labeled cell surface proteins or glycoproteins, is added to each well followed by incubation. Positive wells can be subsequently detected by autoradiography. Positive clones can be grown in bulk culture and supernatants are subsequently purified over a Protein A column (Pharmacia).

The monoclonal antibodies for the pTarg component can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

The monoclonal antibodies and fragments thereof employed as the pTarg component include chimeric antibodies, e.g., humanized versions of murine monoclonal antibodies. Such humanized antibodies may be prepared by known techniques, and offer the advantage of reduced immunogenicity when the antibodies are administered to humans. In one embodiment, the humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May 1993). Procedures to generate antibodies transgenically can be found in GB 2,272,440, U.S. Pat. Nos. 5,569,825 and 5,545,806 and related patents claiming priority therefrom, all of which are incorporated by reference herein.

In a further embodiment of the invention, the targeting part (pTarg) of a cytotoxic chimeric polypeptide is a tumor homing peptide. Such a tumor homing peptide include any homing sequence described by Ellerby et al., in example V, VI, VII, VIII of PCT/US00/01602, the entire disclosure of which is relied upon and incorporated by reference herein.

In preferred embodiments of the invention, the chimeric polypeptide has the sequence CNGRCGG-HFRIGCRHSRIG, or CNGRCGG-D[HFRIGCRHSRIG], or CNGRCGG-Vpr52-96, or CNGRCGG-DKRTQFWYFPGN, or CNGRCGG-D[DKRTQFWYFPGN], or ACDCRGDCFCGG-HFRIGCRHSRIG, or ACDCRGDCFCGG-D[HFRIGCRHSRIG], or ACDCRGDCFCGG-Vpr52-96, or ACDCRGDCFCGG-DKRTQFWYFPGN, or ACDCRGDCFCGG-[DKRTQFWYFPGN], or M350/ScFv-HFRIGCRHSRIG, or M350/ScFv-D[HFRIGCRHSRIG] or M350/ScFv-Vpr52-96, or M350/ScFv-DKRTQFWYFPGN, or or M350/ScFv-D[DKRTQFWYFPGN].

Chimeric polypeptides of the invention can be generated by a variety of conventional techniques. Such techniques include those described in B. Merrifield, Methods Enzymol, 289:3-13, 1997; H. Ball and P. Mascagni, Int. J. Pept. Protein Res. 48:31-47, 1996; F. Molina et al., Pept. Res. 9:151-155, 1996; J. Fox, Mol. Biotechnol. 3:249-258, 1995; and P. Lepage et al., Anal. Biochem. 213: 40-48, 1993.

Peptides can be synthesized on a multi-channel peptide synthesizer using classical Fmoc-based and pseudopeptide synthesis. In one embodiment of the invention, Vpr52-96, Vpr71-96 and Vpr 71-82 and all the Tox, Save and TARG peptides described in Table I, II, III, are synthesized by solid phase peptide chemistry. After cleavage from the resin, the peptides are purified and analyzed by reverse-phase HPLC. The purity of the peptides is typically above 98% according to HPLC trace. The integrity of each peptide can be controlled by matrix Assisted Laser Desorption Time of Flight spectrometry. To avoid rapid degradation of the peptides in biological fluids, one or several amide bonds could be advantageously replaced by peptide bond isosters like retro-inverso (NH—CO), methylene amino (CH₂—NH), carba (CH₂—CH₂) or carbaza (CH₂—CH₂—N(R)) bonds.

Alternatively, the chimeric polypeptides of the invention can be prepared by subcloning a DNA sequence encoding a desired peptide sequence into an expression vector for the production of the desired peptide. The DNA sequence encoding the peptide is advantageously fused to a sequence encoding a suitable leader or signal peptide. Alternatively, the DNA fragment may be chemically synthesized using conventional techniques. The DNA fragment can also be produced by restriction endonuclease digestion of a clone of, for example HIV-1, DNA using known restriction enzymes (New England Biolabs 1997 Catalog, Stratagene 1997 Catalog, Promega 1997 Catalog) and isolated by conventional means, such as by agarose gel electrophoresis.

In another embodiment, the well known polymerase chain reaction (PCR) procedure can be employed to isolate and amplify a DNA sequence encoding the desired protein or peptide fragment. Oligonucleotides that define the desired termini of the DNA fragment are employed as 5′ and 3′ primers. The oligonucleotides can contain recognition sites for restriction endonucleases, to facilitate insertion of the amplified DNA fragment into an expression vector. PCR techniques are described in Saiki et al., Science 239:487 (1988); Recombinant DNA Methology, Wu et al., eds., Academic Press, Inc., San Diego (1989), p.189-196; and PCR Protocols: A Guide to Methods and Applications, Innis et al., eds, Academic Press., (1990). It is understood of course that many techniques could be used to prepare polypeptide and DNA fragments, and that this embodiment in no way limits the scope of the invention.

Several methods can be used to link TARG to TOX and TARG to SAVE, depending on the particular chemical characteristics of the molecules. For example, methods of linking haptens to carrier proteins as used routinely in the field of applied immunology. In one embodiment, a premade a PTPC regulatory molecule (TOX or SAVE) can be conjugated to an antibody as antibody fragment (pTarg) using, for example, carbodiimide conjugation. Carbodiimides comprise a group of compounds that have the general formula R—N+C=N—R, where R and R can be aliphatic or aromatic, and are used for synthesis of peptide bonds. The preparative procedure is simple, relatively fast, and is carried out under mild conditions. Cardodiimide compounds attack carboxylic groups to change them into reactive sites for free amino groups. Carbondiimide conjugation has been used to conjugate a variety of compounds for the production of antibodies.

The water soluble carbodiimide, 1-ethyl-3-(3-dimethylaminopropyl) carbodiimide (EDC) can be useful for conjugating a PTPC regulatory molecule (TOX or SAVE) to an antibody or antibody fragment molecule. Such conjugation requires the presence of an amino group, which can be provided, for example, by a PTPC regulatory molecule (TOX or SAVE), and a carboxyl group, which can be provided by an antibody or antibody fragment.

In addition to using carbodiimides for the direct formation of peptide bonds, EDC also can be used to prepare active esters, such as N-hydroxysucinimide (NHS) ester. The NHS ester, which binds only to amino groups, then can be used to induce the formation of an amide bond with the single amino group of the oxorubicin. The use of EDC and NHS in combination is commonly used for conjugation in order to increase yield of conjugate formation.

Other methods for conjugating a PTPC regulatory molecule (TOX or SAVE) to an antibody or antibody fragment also can be used. For example, sodium periodate oxidation followed by reductive alkylation of appropriate reactants can be used, as can glutaraldehyde crosslinking. However, it is recognized that, regardless of which method of producing a chimeric polypeptide of the invention is selected, a determination must be made that an antibody or antibody fragment maintains its targeting ability and that a PTPC regulatory molecule (TOX or SAVE) maintains its activity.

The chimeric polypeptide of the invention may further incorporate a specifically non-cleavable or cleavable linker peptide functionally interposed between the PTPC regulatory molecule (TOX or SAVE) (pTarg) and the antibody or antibody fragment (pTox). Such a linker peptide provides by its inclusion in the chimeric construct, a site within the resulting chimeric polypeptide that may be cleaved in a manner to separate the intact PTPC regulatory molecule (TOX or SAVE) from the intact antibody or antibody fragment. Such a linker peptide may be, for instance, a peptide sensitive to thrombin cleavage, factor X cleavage, or other peptidase cleavage. Alternatively, where the chimeric polypeptide lacks methionine, the antibody or antibody fragment may be separated by a peptide sensitive to cyanogen bromide treatment. In general, such a linker peptide will describe a site, which is uniquely found within the linker peptide, and is not found at any location in either of the TARG, TOX or SAVE fragment constituting the chimeric polypeptide.

Compositions comprising an effective amount of a chimeric polypeptide of the present invention, in combination with other components, such as a physiologically acceptable diluent, carrier, or excipient, are provided herein. The chimeric polypeptide can be formulated according to known methods used to prepare pharmaceutically useful compositions. They can be combined in admixture, either as the sole active material or with other known materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16^{th} ed. 1980, Mack Publishing Company, Easton, Pa.

In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance, and are thus chosen according to the intended application.

The compositions of the invention comprising the chimeric polypeptide can be administered in any suitable manner, e.g., topically, parenterally, or by inhalation. The term “parenteral” includes injection, e.g., by subcutaneous, intravenous, or intramuscular routes, also including localized administration, e.g., at a site of disease or injury. Sustained release from implants is also contemplated. One skilled in the pertinent art will recognize that suitable dosages will vary, depending upon such factors as the nature of the disorder to be treated, the patient's body weight, age, and general condition, and the route of administration. Preliminary doses can be determined according to animal tests, and the scaling of dosages for human administration is performed according to art-accepted practices.

Compositions comprising nucleic acids in physiologically acceptable formulations are also contemplated. DNA may be formulated for injection, for example.

In one of its most general applications, the invention relates to a recombinant vector incorporating a DNA segment having a sequence encoding the chimeric polypeptide of the invention. For the purposes of the invention, the term “chimeric polypeptide” is defined as including any polypeptide where at least a portion of a viral apoptotic peptide is coupled to at least a portion of an antibody or antibody fragment. The coupling can be achieved in a manner that provides for a functional transcribing and translating of the DNA segment and message derived therefrom, respectively.

The vectors of the invention will generally be constructed such that the chimeric polypeptide encoding sequence is positioned adjacent to and under the control of an effective promoter. In certain cases, the promotor will comprise a prokaryotic promoter where the vector is being adapted for expression in a prokaryotic host. In other cases, the promoter will comprise a eukaryotic promoter where the vector is being adapted for expression in a eukaryotic host. In the later cases, the vector will typically further include a polyadenylation signal position 3′ of the carboxy-terminal amino acid, and within a transcriptional unit of the encoded chimeric polypeptide. Promoters of particular utility in the vectors of the invention are cytomegalovirus promoters and baculovirus promoters, depending upon the cell used for expression. Regardless of the exact nature of the vector's promoters, the recombinant vectors of the invention will incorporate a DNA segment as defined below.

A recombinant host cell is also claimed herein, which incorporates a vector of the invention. The recombinant host cell may be either a eukaryotic cell or a prokaryotic host cell. Where a eukaryotic cell is used, a Chinese Hamster Ovary (CHO) cell has utility. In another embodiment, when used in combination with a baculovirus promoter, the insect cell lines SF9 or SF21 can be used.

This invention will be described in greater detail in the following Examples.

### EXAMPLE 1

### Obtaining the Murine Monoclonal Antibody (Ac M350)

Human fetal cells were chosen as a source of immunization. It was the well-known similarities between fetal and tumoral antigens which inspired us to use fetal cells as a source of immunization to produce monoclonal antibodies directed against the epitopes present on tumoral cells. Oncofetal antigens are glycoproteins which are present during intra-uterine life; they disappear at birth and can be re-expressed in pathological situations, particularly in malignant tumors. There are many examples of this antigen community, the best known models being fetoprotein which is associated with 70% of liver tumors, and <<embryo tumor antigens>>, which is often used in human clinical practice and which is a monitoring parameter for patients suffering from cancers of the digestive tract.

A. M350 Clone Production

These fetal cells were obtained from the sterile removal of the mammary buds of 25-week old female fetuses. Once the buds had been mechanically dissociated into 0.5 mm³ fragments, the cells were resuspended in a Dulbecco medium modified with collagenase and hyalurodinase at 37° C. and shaken for between 30 minutes and 4 hours after being monitored under the microscope. As soon as organoids appear, the cells were deposited onto Ficoll, washed, then cultured in a calcium-free DMEM-F12 medium, in hepes, insulin, choleric toxin, cortisol. Once the cells were subcultured once a week. Using this technique the cells duplicated 10 to 20 times giving sufficient cells for immunization purposes.

Balb/c mice were immunized four times, intraperiotonaly. The fusion was achieved according the classical technic of Kohler and Milstein. The screening was done with fetal mammary cells, adult mammary cells and breast tumors. Several clones appeared and one, M350 clone, was particularly tested on breast tumors and normal breast tissues. 150 tumor sections were tested: (i.e.) infiltrating intra-canalar and intra-lobular adenocarcimonas, infiltrating lobular adenocarcimonas. Tests were performed using an immunoenzymatic technic with alkaline phosphatase. All the tumors tested positive whereas the normal tissues taken from mammary samples tested in parallel were negative for weakly positive. Each slide of normal tissue contained lobular type epithelial structures and cavities inside the paleal tissue.

B. Other Hybridomes

Obtaining new murine monoclonal antibodies against associated breast tumor antigens.

In this technology, C57/B16 mice were immunized four times, intraperitonaly, with a mixture of three different breast tumor cell lines (MCF7, MDA, ZR75-1). After fusion and screening the specificity was studied on normal breast tissues and malignant tumors, other tumor samples and peripheral blood cells. The Monoclonal antibodies showing surface tumor labeling were chosen.

### EXAMPLE 2

A Cell Lines and Viruses

The insert cells derived from ovarian tissue of Spodoptera frugiperda (Sf9 insect cells, Vaughn et coll., 1977) and insect cells derived from Trichoplusia ni (High Five insect cells) were maintained at 28° C. in TC100 medium supplemented with 5% fetal calf serum and were used for the propagation of recombinant baculoviruses and for the production of recombinant proteins. The recombinant baculoviruses are obtained after co-transfection of insect cells with baculovirus viral DNA (Baculogold, Pharmingen) and recombinant transfer vector DNA.

B. Recombinant Transfer Vector: pVL-PS-gp671

The recombinant transfer vector pVL-PSgp671 derived from transfer vector pVL1392 (Invitrogen) is used as transfer vector to generate recombinant viruses. It includes from 5′ to 3′: the peptide signal sequence of gp67 baculovirus glycoprotein, the sequence coding for a His(6)-Tag, the recognition sequence for the Xa Factor, a polylinker region for subcloning the scFv sequence, a link-sequence: GGC required for the covalent association between cytotoxic peptides and ScFv.

The signal peptide sequence from gp67 was added by insertion of a PCR product of gp67 (obtained by PCR from a commercial pcGP67-B plasmid as a template and the PSgp67—Back and PSgp67—For as primers) at the Bg/II site of the pVL1392 plasmid. The sequence coding for the His(6)-Tag sequence and the recognition sequence for the Xa factor were then added by using insertion of oligonucleotides at the 3′ end of the gp67 sequence. By the same way the sequence of the peptide motif required for the covalent association between cytotoxic peptides and ScFv: (-Gly-Gly-Cys) was added at the 3′ part of the polylinker (the first G is encoded by the last nucleotide of the Xmal site).

Insertion at BamH1 and Bg1I of overlaping primers:

Th1: GAT CCC ATC ATC ACC ACC ACC AC (BamHI-His(6))

Th2: ATT GAA GGA AGA GAATTC CCATG (Factor Xa cleveage-EcoRI-NcoI)

Th3: GCT GCA GCC CGG GGG ATG TTA AA (Pst1-XmaI-GGS-STOP-BamHI)

Th4: CTT CCT TCA ATG TGG TGG TGG TGA TGA TGG (link beween Th1 Th2)

Th5: GGG CTG CAG CCA TGG GAA TTC T (link between Th2 and Th3)

Th6: GAT CTT TAA CAT CCC CC (link between Th3 and pVL, -pg67)

C Synthesis of ScFv DNA Fragment

VH and VL Regions of M350:

Total RNA isolated from M350 hybridome have been used as a template for a reverse transcription using oligo (dT) as primers (Reverse Transcription IBI Fermentas). A PCR realized with those cDNAs and specific primers (mouse Ig-Prime-Kit, Novagen) have led to the selective amplification of VH and VL chains. These regions are then cloned in “blunt” in pST-Blue 1 plasmid and sequenced.

VH and VL Regions of Other Hybridoines:

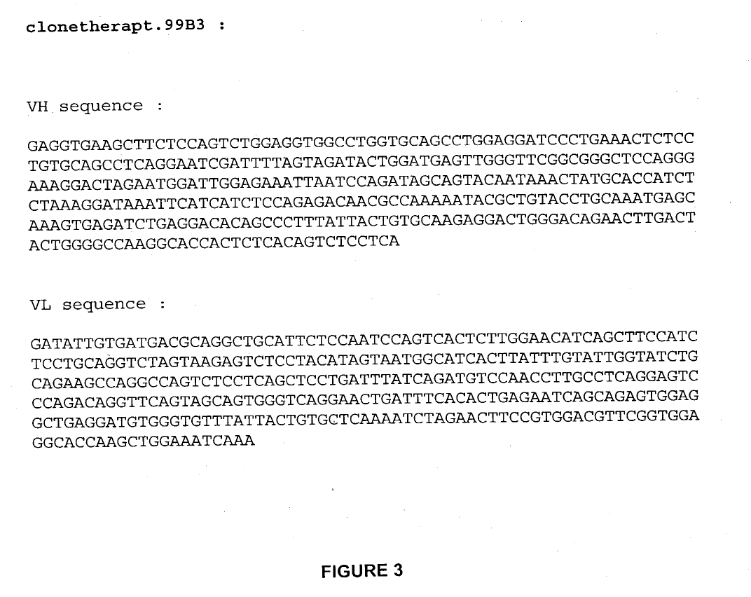

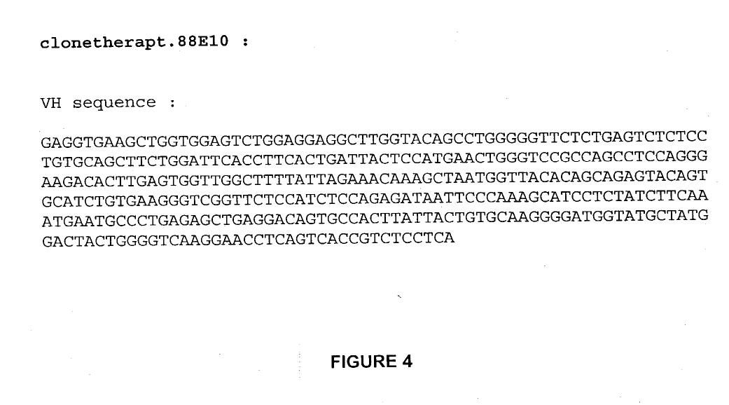

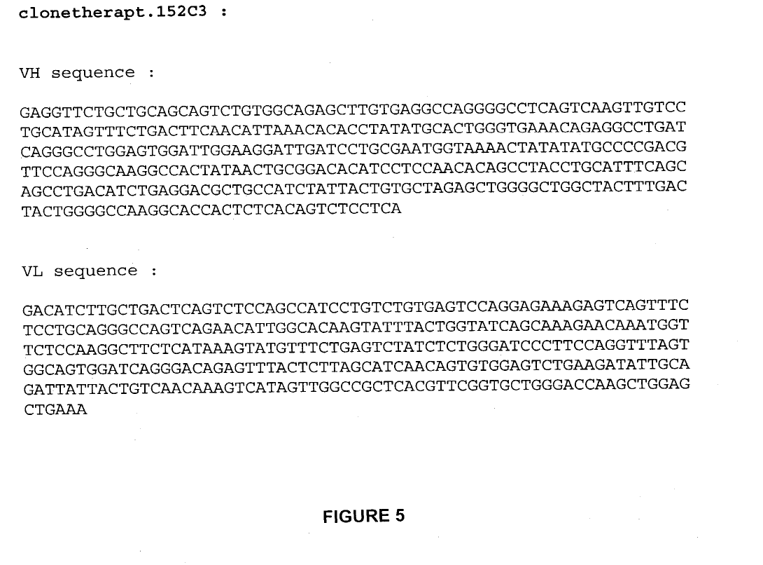

Total RNA isolated from selected hybridome was used as a template for a reverse transcription using oligo (dT) (Reverse Transcription IBI Fermentas). A PCR with specific primers (mouse Ig-Prime-Kit, Novagen) led to the selective amplification of VH and VL chains. These products are then cloned in pGEMT (TA cloning System front PROMEGA) vector and sequenced. Three new VH and VL sequences were determined from clone therap.99B3 (FIG. 3), clone therap.88E10 (FIG. 4), and therap.152C3 (FIG. 5).

Obtention of the ScFv-transfer Vector:

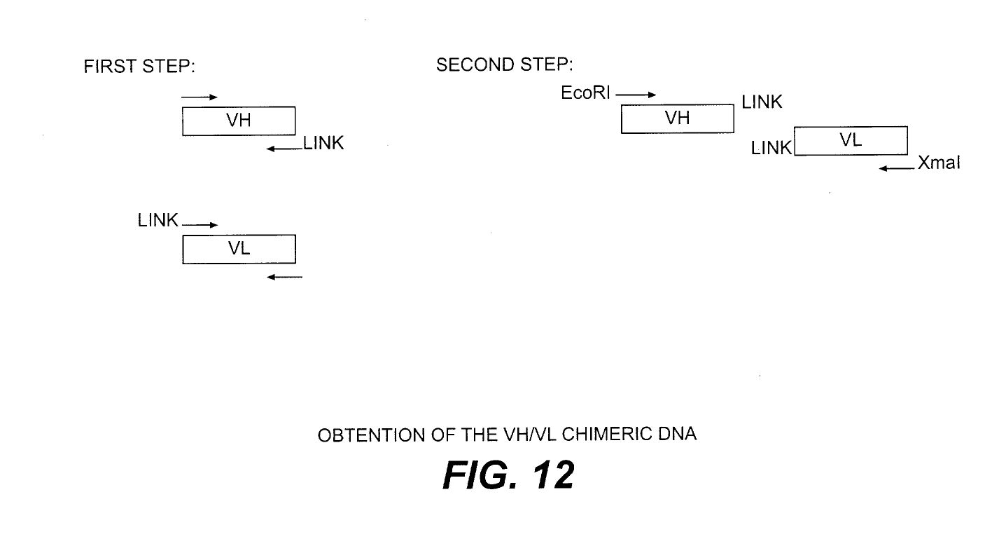

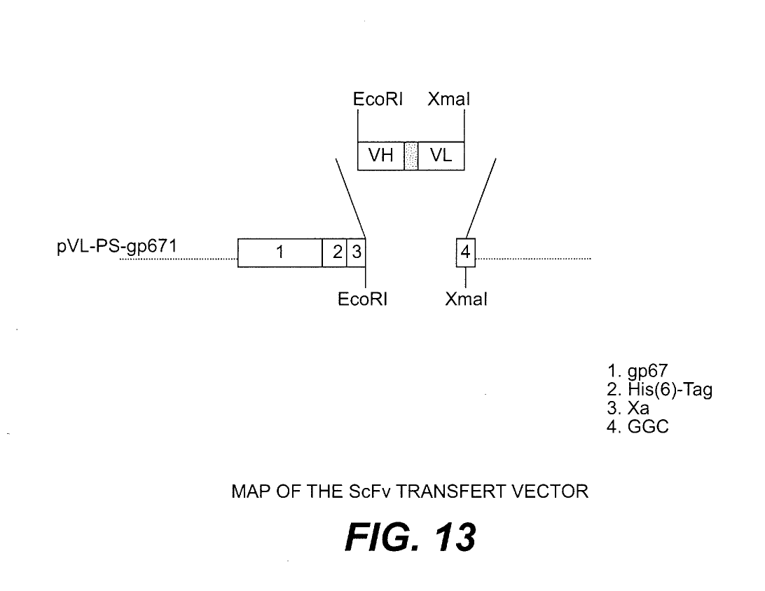

VH-link-VL chimeric DNA were done by fusion-PCR in two steps (FIG. 12). The first step added a link-sequence (Gly-Gly-Gly-Gly-Ser) at the 3′ of the VH chain and at the 5′ end of the VL chain respectively. The second step was a PCR fusion leading to the chimeric DNA: VH-link-VL. The set of primers used in this second step brings a 5′-EcoRI and a 3′-XmaI sites to VH and VL respectively that will be used for the subcloning of the final product in pVL-PSgp671 vector (FIG. 13).

D Cotransfection and Purification of Recombinant Baculoviruses

Sf9 cells were cotransfected with viral DNA (BaculoGold ; Pharmingen) and recombinant transfer vector DNA (pVL-PSgp671-ScFv) by the lipofection method (Feloner and Ringold, 1989) (DOTAP; Roche). Screening and purification of recombinant viruses were carried out by the common procedure described by Summers and Smith (Summers and Smith, 1987). The recombinant virus was named BAC-PSgp671-scFv and amplified to constitute a viral stock with an M0I of 10⁸.

E Analysis of Recombinant Proteins

Infected cells were collected, washed with cold phosphate-buffered saline (PBS) and resuspended in sample reducing buffer (Laemmli, 1970). After boiling (100° C. for 5 min), proteins samples were resolved by 12.5% sodium dodecyl sulfate-polyacrylamide gel electrophoresis (SDS-PAGE) under denaturing conditions (Laemmli, 1970). The apparent molecular weight of the protein was check by coomassie blue staining or the proteins were transferred onto a nitrocellulose filter (Schleicher and Schuell BAS 85, 0.45 μm) with a semidry blotter apparatus (Ancos). The nitrocellulose membrane was then stained with Ponceau Red (Sigma) and subsequently blocked with a solution of Tris-saline buffer (0.05 M Tris-HCI ph7.4, 0.2 M NaCl) containing 0.05% Tween 20 and 5% non fat milk (TS-sat). ScFv was detected using a mouse monoclonal antibody raised against His(6)-Tag (SIGMA) as primary antibody and a sheep anti-mouse immunoglobulin G (IgG)-horseradish peroxydase conjugate as secondary antibody (1; 3000 Amersham). The immunoreactive bands were visualized by using ECL reagents as described by the manufacturer (Amersham).

F Protein Production and Purification

To obtain viral stock, Sf9 insect cells cultured in IPL41 medium and 5% FCS are infected in exponential phase with the recombinant baculoviruses at MOI1. After a 7-day incubation period at 28° in IPL41 medium with 5% FCS, the supernatant is harvested by centrifugation at 8000 RPM during 15 min. Then High-five insect cells cultured in Xpress media (Biowhitaker) are infected with recombinant baculovirus in exponential phase at MOI 10, following lh30 of infection High Five cells were harvested by centrifugation and resuspended in Xpress media without serum. After a 4-day period of incubation at 28° C., the supenatant is harvested by centrifugation at 8000 RPM during 15 min. These supernatants are then concentrated by two rounds of ammonium sulfate precipitation. The precipitate obtained by sedimentation is dialyzed during 12 hours and purified using batch of Ni—NTA agarose beads as described by the manufacturer (Qiagen). After dialysis (2 days, PBS, 4° C.) and analysis by Coomassic staining purified proteins were used for the covalent association with cytotoxic peptides.

### EXAMPLE 3

Method of Coupling ScFv to pTox

The peptide was assembled using Fmoc solid phase peptide synthesis, after the last Fmoc deprotection a propionyloxy succinimide ester was allowed to react, in the presence of diisopropyl ethylamine, with the alpha amino group of the peptide. At the end of the reaction (30 min) the peptide resin was washed with methylene chloride and the peptide was classically cleaved and deprotected under acidic conditions. The activated peptide was then purified by HPLC and its integrity was confirmed by mass spectrometry. The activated peptide was then allowed to react with the ScFv with peptide in a molar ratio of 10:1 (pH7, PBS, glass tube over agitation for 3 hours at room temperature). Then, dialysis was done for 48 h against PBS a 4° C. Four Tox peptides were coupled to ScFv using this method:

| | |
|---|---|
| Tox 11 | ScFv-M350-Jac5 (Vpr71-96[C761]) |
| Ctr1ToX11I | ScFv-M350-Jac5M (Vpr71-96[C76S;R73,80A]) |
| Tox 12 | ScFv-Vpr52-96[C76S] |
| Ctr1Tox12 | ScFv-Vpr52-96[C76S;R73A;R80A] |

### EXAMPLE 4

Examples of Targ-Tox or Targ-Save Structures

All the Tox peptides can have a facultative N-terminal biotin and a facultative C-terminal amide fonction. Tox0 is a Tox peptide which does not necessarily require an association with a Targ. Tox1, Tox2, Tox 5, Tox6, Save1, Save2 and their respective control can posses a facultative gly-gly-(-GG-) linker between the Targ and the Tox/Save motif

| | |
|---|---|
| Tox0 | Biot-DTWTGVEALIRILQQLLFIHFRIGCRHSRIGIIQQRRTRNGASKS |
| Ctr1Tox0 | Biot-DTWTGVEALIRILQQLLFHFAIGCRHSAIGIIQQRRTRNGASKS |
| Tox1 | Biot- CNGRC-GG-HFRIGCRHSRIG |
| Ctr1Tox1 | Biot- CNGRC-GG-HFAIGCRHSAIG |
| Ctr2Tox1 | Biot-CNGRC-GG-CNGRC |
| Ctr3Tox1 | Biot-GG-HFRIGCRHSRIG |
| Ctr4Tox1 | Biot-CNGRC-GG-Scramble |
| Ctr5Tox1 | Biot-KETWWETWWTEW-GG-HFRIGCRHSRIG |
| Tox2 | Biot-ACDCRGDCFC-GG-HFRIGCRHSRIG |
| Ctr1Tox2 | Biot- ACDCRGDCFC-GG-HFAIGCRHSAIG |
| Tox5 | |
| Tox5 | Biot-CNGRC-GG-DKRTQFWRYFPGN (hANT2m) |
| Ctr1Tox5 | Biot-CNGRC-GG-DKRTQFWRYFAGN (hANT2) |
| Ctr2Tox5 | Biot-CNGRC-GG-DRHKQFWRYFPGN (hANT1m) |
| Ctr3Tox5 | Biot-CNGRC-GG-DKHTQFWRYFPGN (hANT3m) |
| Ctr4Tox5 | Biot-GG-DKRTQFWRYFPGN (hANT2m) |
| Ctr5Tox5 | Biot-GG-DRHKQFWRYFPGN (hANT1m) |
| Ctr6Tox5 | Biot-GG-DKHTQFWRYFPGN (hANT3m) |
| Ctr7Tox5 | Biot-CNGRC-GG-Scramble |
| Tox6 | |
| Tox6 | Biot-ACDCRGDCFC-GG-DKRTQFWRYFPGN (hANT2m) |
| Ctr1Tox6 | Biot-ACDCRGDCFC-GG-DKRTQFWRYFAGN (hANT2) |
| Ctr2Tox6 | Biot-ACDCRGDCFC-GG-DRHKQFWRYFPGN (hANT1m) |
| Ctr3Tox6 | Biot-ACDCRGDCFC-GG-DKHTQFWRYFPGN (hANT3m) |
| Ctr4Tox6 | Biot-ACDCRGDCFC-GG |
| Ctr5Tox6 | Biot-ACDCRGDCFC-GG-Scramble |
| Tox 11 | |
| Tox 11 | ScFv-M350-Jac5(Vpr71-96[C76]) |
| Ctr1Tox11 | ScFv-M350-Jac5M(Vpr71-96[C76;R73,80A]) |
| Save1 | |
| Save1 | Biot-RKKRRQRRR-DKRTQFWRYFAGN (hANT2) |
| Ctr1Save1 | Biot-RKKRRQRRR-DKRTQFWRYFPGN (hANT2m) |
| Ctr2Save1 | Biot-RKKRRQRRR-DRHKQFWRYFAGN (hANT1) |
| Ctr3Save1 | Biot-RKKRRQRRR-DKHTQFWRYFAGN (hANT3) |
| Ctr4Save1 | Biot-RKKRRQRRR |
| Ctr5Save1 | Biot-RKKRRQRRR-Scramble |
| Save2 | |
| Save2 | Biot-RKKRRQRRR-LASGGAAGATSLCFVYPL (hANT[117-134]) |
| Ctr1Save2 | Biot-RKKRRQRRR-GAWSNVLRGMGGAFVLVLY (ANTTM6[271-289]) |
| Ctr2Save2 | Biot-RKKRRQRRR-scramble |

### EXAMPLE 5

### Evaluation of Mitochondrial and Nuclear Parameters of Apoptosis in Cells (Cell Lines) and Cell-free Systems

A. Cells

MCF-7, MDA-MB231, COS and HeLa cells are cultured in complete culture medium (DMEM supplemented with 2 mM glutamine, 10% FCS, 1 mM Pyruvate, 10 mM Hepes and 100 U/ml pencillin/streptomycin). Jurkat cells expressing CD4 and stably transfected with the human Bcl-2 gene or a Neomycin (Neo) resistance vector [Aillet, et al., 1998 J. Virol. 72:9698-9705] only were kindly provided by N. Israel (Pasteur Institute, Paris). Neo and Bcl-2 U937 cells [Zamzami et al., 1995 J. Exp. Med], and CEM-C7 cells are cultured in RPMI 1640 Glutamax medium supplemented with 10% FCS, antibiotics, and 0.8 μg/ml G418.

The cell tests that have been implemented determine the pathway (intracellular penetration, then subcellular localization) of the candidates, and the apoptotic status (ΔΨm, activation and relocalization of cell death effectors, content in nuclear DNA) of the target cell. In order to determine these parameters it is necessary to use fluorescent probes to label the cells and/or the candidates molecules and to implement the following two analytical procedures: multi-parameter cytofluorimetry and fluorescent microscopy. As far as neuroprotection is concerned, tests were carried out on primary cultures of cortical neuronal cells from mice embryos. As far as cardioprotection is concerned, tests were carried out on primary cultures of cardiomyocytes from mice embryos.

Intra-cellular pathway tests: the TARG-TOX ou TARG-SAVE peptides coupled either with biotin (detected using fluorochromes conjugated with streptavidin; or by ligand-blot after subcellular fractioning) or with FITC (detected by direct observation of living cells, videomicroscopy and image analysis) are added to the cells. It possible to favor the TOX or SAVE mitochondrial routing by inserting mitochondrial addressing signals (the Apoptosis Inducing Factor or ornithin transcarbamylase, for example). Similarly, the mitochondrial routing is evaluated after modifying sequences and certain lateral chains (phosphorylations, methylations), then replacing the peptides by peptidomimetics.

Multi-parameter analysis of apoptosis on tumoral and endothelial cell lines, and primary neurons. Fluorescents probes wil be used to mesure the state of the mitochondrial transmembrane potential (JCI, DioC6, mitoTrackers) and nuclear condensation (Hoescht). Similarly, the post-mitochondrial parameters of apoptosis are evaluated using classical hypoploidy tests and cell surface labeling with annexin V—FITC.

In this type of tests, we evaluate either the cytotoxic potential of the TARG-TOX, i.e. their capacity to kill (via a mitochondrial effect) tumoral ou endothelial cell lines (the best TARG-TOX must also kill over-expressing Bel-2 cell lines); or the cytoprotective potential of the TARG-SAVE when the neurons are subjected to different apoptogenic treatments.

B. Apoptosis Modulation

PBS-washed cells (1-5×10⁵/ml) are incubated with (1 to 5 μM) of pTarg-pTox in complete culture medium supplemented or not with cyclosporin A (CsA; 1 μM), bongkrekic acid (BA; 50 μM), and/or the caspase inhibitors N-benzyloxycarbonyl-Val-Ala-Asp.fluoromethylketone (Z-VAD.fmk; 50 μM; Bachem Bioscience, Inc.), Boc-Asp-fluoromethylketone (Boc-D.fmk), or N-benzyloxycarbonyl-Phe-Ala-fluoromethylketone (Z-FA.fmk; all used at 100 μM added each 24 h; Enzyme Systems). During exposure to pTarg-pTox, human primary PBLs from healthy donors, purified with Lymphoprep (Pharmacia), are cultured in RPMI 1640 Glutamax medium without any addition of serum. In contrast, PHA blasts (24 h of 1 μg/ml PHA-P [Wellcome Industries]; 48 h with 100 U/ml human recombinant IL-2 [Boehringer Mannheim]) are cultured with 10% FCS.

C. Cytofluorimetric Determinations of Apoptosis-associated Alterations in Intact Cells

For cytofluorometry, the following fluorochromes are employed: 3,3′-dihexyloxacarbo-cyanine iodide (DiOC(6)3; 40 nM) for mitochondrial transmembrane potential (ΔΨm) quantification, hydroethidine (4 μM) for the determination of superoxide anion generation, and propidium. iodide (PI; 5 μM) for the determination of viability (Zamzami, N., et al., 1995. J. Exp. Med. 182:367-377). The frequency of subdiploid cells is determined by PI (50 μg/ml) staining of ethanol-permeabilized cells treated with 500 μg/ml RNase (Sigma Chemical Co.; 30 min, room temperature [RT]) in PBS, pH 7.4, supplemented with 5 mM glucose (Nicoletti, I. et al., 1991. J. Immunol. Methods. 139:271-280).

D. Fluorescence Staining of Life Cells and Immunofluorescence

For the assessment of mitochondrial and nuclear features of apoptosis, cells cultured on a cover slip are incubated with the ΔΨm-sensitive dyes chloromethyl-X-rosamine (CMXRos; 50 nM; Molecular Probes, Inc.) or 5,5′,6,6′-tetrachloro-1,1′,3,3′-tetraethylbenzimidazolylcarbocyanine iodide (JC-1, 2 μM, Molecular Probes), the ΔΨm-insensitive dye Mitotracker green (1 μM; Molecular Probes, Inc.), and/or Hoechst 33342 (2 μM, Sigma) for 30 min at 37° C. in complete culture medium (.Marzo, Iet al. 1998. Science. 281:2027-2031).

E. For in situ Determinations of pTarg-pTox Internalisation

For in situ determinations of TARG-(MLS)-TOX/SAVE internalisation, cells are incubated at different times with TARG-(MLS)-TOX/SAVE, and then cells are fixed with 4% paraformaldehyde and 0.19% picric acid in PBS (pH 7.4) for 1 h at RT. Fixed cells are permeabilized with 0.1% SDS in PBS at RT (for 5 min), blocked with 10% FCS, and stained with an mAb specific for hexa-histidine tag (clone HIS-1, IgG2a, SIGMA) revealed by a goat anti-mouse PE conjugate [Southern Biotechnology Associates, Inc.]), Hsp60 (mAb H4149 [Sigma Chemical Co.], revealed by a goat anti-mouse IgG1 FITC conjugate), cytochrome c oxidase (COX; mAb 20E8-C12 [Molecular Probes, Inc.], revealed by a goat anti-mouse IgG2a FITC conjugate), or when the Targ is a biotinylated peptide, a streptavidin-PE reagent is added 30 min. followed by detection of the fluorescence intensity by fluorescence (and/or confocal) microscopy.

F. Assessment of Mitochondrial Parameters in vitro

Mitochondria are purified from rat liver, as described (Costantini et al., 1996), and resuspended in 250 mM sucrose+0.1 mM EGTA+10 mM-tris[hydroxymethyl]methyl-2-Aminoethanesulfonic acid, pH=7.4). For the induction of PT, mitochondria (0.5 mg protein per ml) are resuspended in PT buffer (200 mM sucrose, 10 mM Tris-MOPS (pH 7.4), 5 mM Tris-succinate, 1 mM Tris-phosphate, 2 μM rotenone, and 10 μM EGTA-Tris), and monitored in an F4500 fluorescence spectrometer (Hitachi, Tokyo, Japan) for the 90° light scattering of light (545 nm) to determine large amplitude swelling after addition of 2 mM atractyloside (Atr), 1 μM cyclosporin A (CsA; Novartis, Basel, Switzerland), 5 μM CaCl₂, and/or 0.5 to 20 μM of pTarg-pTox or pTarg-pSave. For the determination of the ΔΨₘ, mitochondria (0.5 mg protein per ml) are incubated in a buffer supplemented with 1 μM rhodamine 123 (Molecular Probes, Eugene, Oreg.) and the dequenching of rhodamine fluorescence (excitation 505 nm, emission 525 nm) is measured as described (Shimizu et al, 1998). Supernatants from mitochondria (6800 g for 15 min; then 20 000 g for 1 h; 4° C.) are frozen at −80° C. until determination of apoptogenic activity on isolated nuclei, DEVD-afc cleaving activity, and immunodetection of cytochrome c and AIF. Cytochrome c and AIF are detected by means of a monoclonal antibody (clone 7H8.2C12, Pharmingen) and a polyclonal rabbit anti-serum (Susin et al. 1999) respectively.

Swelling of Isolated Mitochondria

TABLE F1
Tox0, Tox1, Tox5, Tox6 induce permeability
transition pore (PTP) openning

| | Induction of Mitochondrial swelling (sw) |
|---|---|
| | +++ rapid sw; ++ low sw; + very low sw; |
| Name of molecules 5 μM | − no sw t 20 min |
| — | − |
| Tox0 | +++ |
| Tox1 | ++ |
| Ctr1 Tox1 | − |
| Ctr2 Tox1 | − |
| Ctr3 Tox1 | + |
| Ctr4 Tox1 | − |
| Tox5 | ++ |
| Tox6 | ++ |

TABLE F2
Save 1 and Save 2 inhibit atractyloside-induced PTP openning

| Name of molecules | Mitrochondrial swelling (sw) % |
|---|---|
| — | 2 |
| Ca 2 + 100 μM | 100 |
| Atractyloside 600 μM | 110 |
| Save I 5 μM | 2 |
| Save I 5 μM + Atr 600 μM | 5 |
| Save I 20 μM | 12 |
| Save I 20 μM + Atr 600 μM | 12 |
| Save II 10 μM | 2 |
| Save II 20 μM | 16 |
| Save II 10 μM + Atr 600 μM | 16 |
| Save II 20 μM + Atr 600 μM | 16 |

G. ANT Purification and Reconstitution in Liposomes

ANT was purified from rat heart mitochondria as previously described (8). After mechanical shearing, mitochondria were suspended in 220 mM mannitol, 70 mM sucrose, 10 mM Hepes, 200 μM EDTA, 100 mM DTT, 0.5 mg/ml subtilisin, pH7.4, kept 8 min on ice and sedimented twice by differential centrifugations (5 min, 500×g, and 10 min, 10,000×g). Mitochondrial proteins were solubilized by 6% [v:v] Triton X-100 (Boehringer Mannheim) in 40 mM K₂HPO₄, 40 mM KCl, 2 mM EDTA, pH 6.0, for 6 min at RT and solubilized proteins were recovered by ultracentrifugation (30 min, 24,000×g, 4° C.). Then, 2 ml of this Triton X-100 extract was applied to a column filled with 1 g of hydroxyapatite (BioGel HTP, BioRad), eluted with previous buffer and diluted [v:v] with 20 mM MES, 200 μM EDTA, 0.5% Triton X-100, pH6.0. Subsequently, the sample was separated with a Hitrap SP column using a FPLC system (Pharmacia) and a linear NaCl gradient (0-1M). Proteins concentration was determined using microBCA-assay (Pierce, Rockfoll, Ill.). Purified ANT and/or recombinant Bcl-2 were reconstituted in PC/cardiolipin liposomes. Briefly, to prepare liposomes, 45 mg PC and 1 mg cardiolipin were mixed in 1 ml chloroform, and the solvent was evaporated under nitrogen. Dry lipids were resuspended in 1 ml liposome buffer (125 mM sucrose+10 mM−2-hydroxyethylpiperazine-N′-2 ethanesulfonic acid; Hepes, pH 7.4) containing 0.3% n-octyl-β-D-pyranoside and mixed by continuous vortexing for 40 min at RT. ANT (0.1 mg/ml) or recombinant Bcl-2 (0.1 mg/ml) were then mixed with liposomes [v:v] and incubated for 20 min at RT. Proteoliposomes were finally dialysed overnight at 4° C.

H. Pore Opening Assay

ANT-proteoliposomes are sonicated in the presence of 1 mM 4-MUP and 10 mM KCl (50W, 22 sec, Branson sonifier 250) on ice as previously described (28). Then, liposomes were separated on Sepadex G-25 columns (PD-10, Pharmacia) from unencapsulated products. 25 μl-aliquots of liposomes were diluted to 3 ml in 10 mM Hepes, 125 mM saccharose, pH 7.4, mixed with various concentrations of the proapoptotic inducers and incubated for 1 h at RT. Potential inhibitors of mitochondrial membranes permeabilization such as BA, ATP and ADP, were added to the liposomes 30 min before treatment. After addition of 10 μl-alkaline phosphatase (5 U/ml, Boehringer Mannheim) diluted in liposomes buffer+0.5 mM MgCl₂, samples were incubated for 15 min at 37° C. under agitation and the enzymatic conversion of 4-MUP in 4-MU was stopped by addition of 150 μl Stop buffer (10 mM Hepes-NaOH, 200 mM EDTA, pH 10). The 4-MU-dependent fluorescence (360/450 nm) was subsequently quantitated (28) using a Perkin Elmer spectrofluorimeter. Atractyloside, a pro-apoptotic permeability transition inducer, was used in each experiment as a standard to determine the 100% response. The percentage of 4-MUP release induced by Vpr-derived peptides or pTarg-ptox was calculated as following: [(fluorescence of liposomes treated by pTar-pTox−fluorescence of untreated liposomes)/(fluorescence of liposomes treated by atractyloside−fluorescence of untreated liposomes)]×100.

ANT Pore Opening Assay:

TABLE H1
examples of fuctionnal interaction between ANT and Tox or Save
constructs. Tox0 and Tox6 induce ANT-protéoliposomes permeabilisation.
Save1 and Save2 block Atractyloside (Atra) -induced ANT-
protéoliposomes permeabilisation

| | Permeabilisation of ANT - |
|---|---|
| | proteoliposomes |
| | +++ high UMP release; ++ UMP release; |
| molecules | + low UMP release; − no UMP release |
| − | − |
| Atra 50 μM | + |
| Atra 100 μM | ++ |
| Atra 200 μM | +++ |
| Tox0 (Biotin-Vpr52-96) 2 μM | +++ |
| Tox6 5 μM | ++ |
| Biotin-Vpr71-96[C76S] 5 μM | ++ |
| Save1 5 μM | − |
| Atra 200 μM + Save1 5 μM | − |
| Save2 5 μM | − |
| Atra 200 μM + Save2 5 μM | − |

I. Binding Assays and Western Blot

Mouse liver mitochondria were isolated as described (zamzami et al., 2000). For the determination of cytochrome C release, supernatants from pTarg-pTox treated mitochondria (6800 g for 15 min; then 20 000 g for 1 h; 4° C.) were frozen at −80° C. until immunodetection of cytochrome c (mouse monoclonal antibody clone 7H8.2CI2, Pharmingen). For binding assays, purified mitochondria were incubated (250 μg of protein in 100 μl swelling buffer) for 30 min at RT 5 μM (binding assay) of pTarg-pTox or biotin-pTarg-pTox. Mitochondria were lysed either after incubation with biotinylated Vpr52-96 (upper panel) or lysed before (lower panel) with 150 μl of a buffer containing 20 mM Tris/HCl, pH 7.6; 400 mM NaCl, 50 mM KCl, 1 mM EDTA, 0.2 mM PMSF, aprotinin (100U/ml), 1% Triton X-100 and 20% glycerol. Such extracts were diluted with 2 volumes of PBS plus 1 mM EDTA before the addition of 150 μl avidin-agarose (ImmunoPure, from Pierce) to capture the biotin-labeled Vpr52-96 complexed with its mitochondrial ligand(s) (2 hours at 4° C. in a roller drum). The avidin-agarose was washed batchwise with PBS (5×5 ml; 1000 g, 5 min, 4° C.), resuspended in 100 μl of 2 fold concentrated Laemmli buffer containing 4% SDS and 5 mM β-mercaptoethanol, incubated 10 min at RT and centrifuged (1000 g, 10 min, 4° C.). Finally, the supernatants were heated at 95° C. for 5 min and analysed by SDS-PAGE (12%), followed by Western blot and immunodetection with a rabbit polyclonal anti-serum against human ANT (kindly provided by Dr. Heide H. Schmid; The Hormel Institute, University of Minnesota, MI; Ref).

J. Flow Cytometric Analysis of Purified Mitochondria

Mouse liver mitochondria are isolated as described (zamzami et al., 2000). Purified mitochondria are resuspended in PT buffer (200 mM sucrose, 10 mM Tris-MOPS (pH 7.4), 5 mM Tris-succinate, 1 mM Tris-phosphate, 2 μM rotenone, and 10 μM EGTA). Cytofluorometric (FACSVantage, Beckton Dickinson) detection is restricted to mitochondria by gating on the FSC/SSC parameters and on the main peak of the FSC-W parameter. Confirmation a posteriori of the validity of these double gating is obtained by labeling of mitochondria with the ΔΨₘ-insensitive mitochondrial dye MitoTracker® Green (75 nM; Molecular Probes; green fluorescence). To determine the percentage of mitochondria having a low ΔΨₘ, the ΔΨₘ-pTox sensitive fluorochrome JC-1 (200 nM; 570-595 nm) is added 10 min before CCCP or pTarg- molecules. Percentage of mitochondria having a lowΔΨₘ, is determined in dot-plot FSC/FL-2 (red fluorescence) windows.

K. Cell-free System of Apoptosis

AIF activity in the supernatant of mitochondria is tested on HeLa cell nuclei, as described (Susin et al., 1997b). Briefly, AIF-containing supernatants of mitochondria are added to purified HeLa nuclei (90 min, 37° C.), which are stained with propidium iodide (PI; 10 μg/ml; Sigma Chemical Co.) and analyzed in an Elite II cytofluorometer (Coulter) to determine the frequency of hypoploid nuclei. In some experiments isolated mitochondria, cytosols from Jurkat or CEM cells (prepared as described (Susin et al., 1997a)), and/or pTarg-pTox are added to the nuclei. Caspase activity in the mitochondrial supernatant was measured using Ac-DEVD-amido-4-trifluoromethylcoumarin (Bachem Bioscience, Inc.) as fluorogenic substrate.

L. Purification and Reconstitution of PTPC in Liposomes

PTPC from Wistar rat brains are purified and reconstituted in liposomes following published protocols (Brenner et al., 1998; Marzo et al., 1998b). Briefly, homogenized brains are subjected to the extraction of triton-soluble proteins, adsorption of proteins to a DE52 resin anion exchange column, elution on a KC1 gradient, and incorporation of fractions with maximum hexokinase activity into phosphatidyl choline/cholesterol (5:1, w:w) vesicles by overnight dialysis. Recombinant human Bcl-2 (1-218) lacking the hydrophobic transmembrane domain (Δ219-239), produced and purified as described (Schendel et al., 1997) are added during the dialysis step at a dose corresponding to 5% of the total PTPC proteins (approximately 10 ng Bcl-2 per mg lipids). Liposomes recovered from dialysis are ultrasonicated. (120 W) during 7 sec in 5 mM malate and 10 mM KCl, charged on a Sephadex G50 columns (Pharmacia), and eluted with 125 mM sucrose+10 mM HEPES (pH 7.4). Aliquots (approx. 10⁷) of liposomes are incubated during 60 min at RT in 125 mM sucrose+10 mM HEPES (pH 7.4) in the presence or absence of pTarg-pTox, [52-96]Vpr or atractyloside. Then, liposomes are equilibrated with 3,3′dihexylocarbocyanine iodide (DiOC₆(3), 80 nM, 20-30 min at RT; Molecular Probes), and analyzed in a FACS-Vantage cytofluorometer (Becton Dickinson, San José, Calif., USA) for DiOC₆(3) retention, as described (Brenner et al., 1998; Marzo et al., 1998b).

Triplicates of 5×10⁴ liposomes are analyzed and results are expressed as % of reduction of DiOC₆(3) fluorescence, considering the reduction obtained with 0.25% SDS (15 min, RT) in PTPC liposomes as 100% value.

Examples of specific peptides and constructs relating to this invention that can be utilized in carrying out the foregoing techniques are shown in Tables I, II, and III, as well as any chimeric molecule that is a combination between TARG and TOX or TARG and SAVE peptides or peptidomimetics.

### EXAMPLE 6

Surface Plasmon Resonance Indicates that Tox0, Tox1, Tox5, Tox6, Save1 Binds Purified ANT but not Purified VDAC.

Methodology.

Sensor Chips SA (streptavidin coated sensor chips) were used for immobilisation of the different peptides. Tox1 was immobilised at a density of 0.7 ng/mm², Tox0 at a density of 3.7 ng/mm²,. Ctr1Tox0 at a density of 1.4 ng/mm², Tox5 at a density of 1 ng/mm², Tox6 at a density of 1 ng/mm2, Save1 at a density of 1.3 ng/mm², and the control peptide at a density of 0.8 ng/mm². Association and dissociation kinetics of ANT and VDAC interactions were followed at a rate of 10 μL/min for 10 minutes (5 minutes association and 5 minutes dissociation). The ligand was regenerated with a 1 minute flux of KSCN 3M. The obtained sensorgrams were analysed by the BlAeval 3.1 software using the method of double references (Myszka D. G. 2000. Kinetic, equilibrium and thermodynamic analysis of macromolecular interactions with BIACORE. Methods Enzymol. 323:325-340). From the sensorgrams with the ligands were first substracted the sensorgrams obtained with the corresponding analyte solvents. A second substraction was performed with the sensorgrams obtained with the control peptide ligand. The control peptide for the Tox and Save peptides was biot-H19C corresponding to the sequence of the β2-adrenergic receptor (Lebesgue D., Wallukat G., Mijares A., Granier C., Argibay J., and Hoebeke J. (1998) An agonist-like monoclonal antibody to the human β 2-adrenergic receptor. Eur.J. Pharmacol. 348:123-133). The control peptide for Tox0 was Ctr1Tox0.

Results.

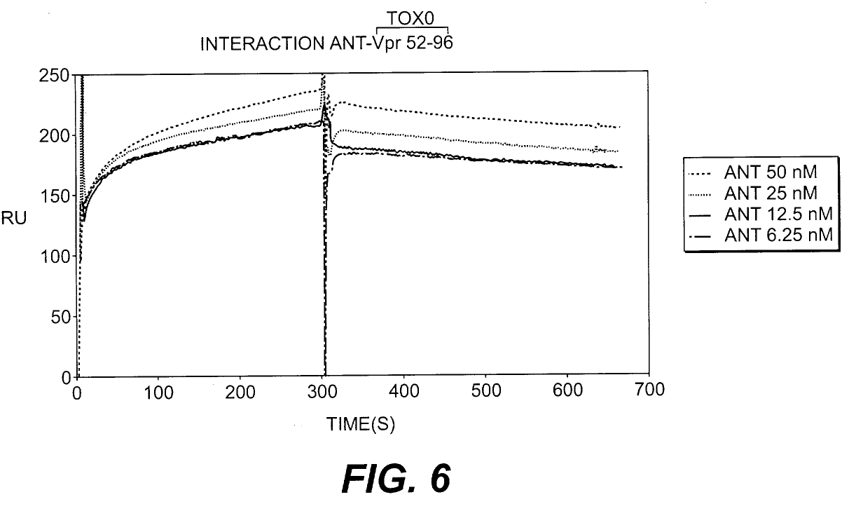

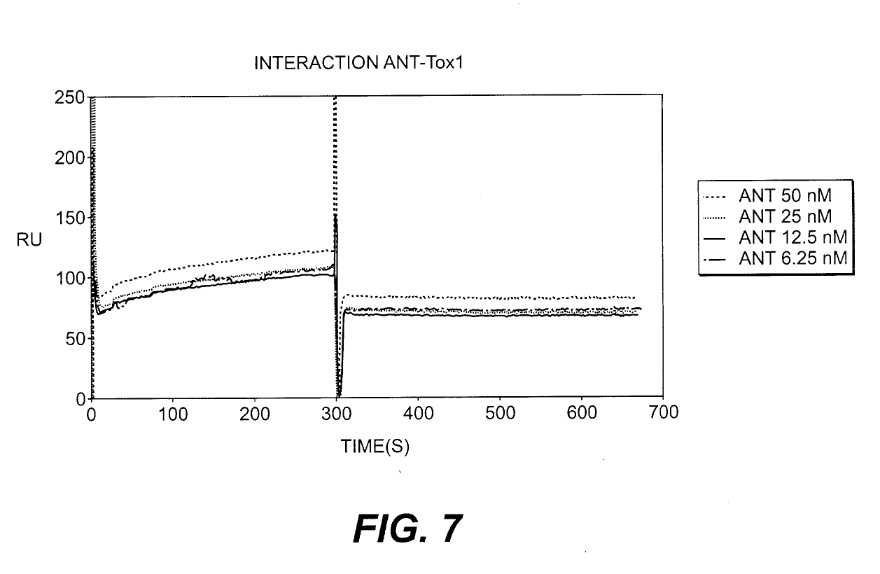

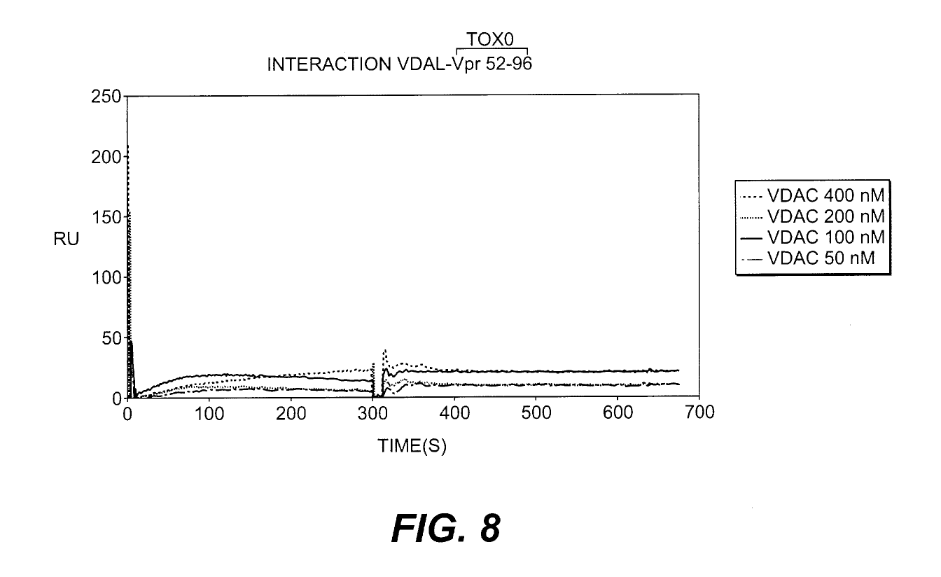

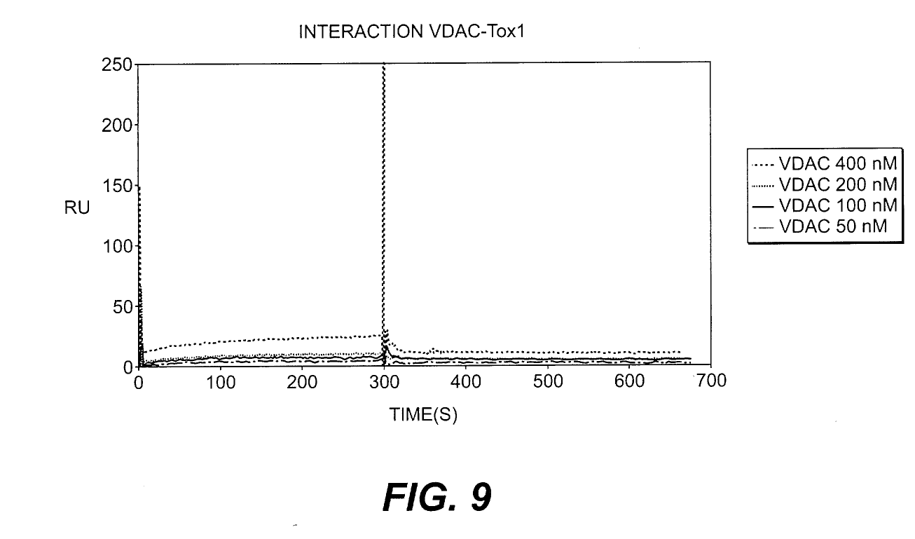

FIG. 6 shows the interaction between ANT and Vpr for 4 ANT concentrations (6.25 to 50 nM). The sensorgrams were best analysed using the simple Lagmuir model with drifting baseline and resulted in a Kd of 0.15 nM with a Rmax of 160 (ₓ2=7.24). The same analysis was performed for the sensorgrams showing the interaction between ANT and Tox1 (FIG. 7). Studying the VDAC interaction both with Tox0 and Tox1 at VDAC concentrations which were ten times higher (FIGS. 8 and 9), the sensorgrams showed only extremely low association with the peptide ligand and the obtained curves could not be analysed by the different Langmuir bindings models.

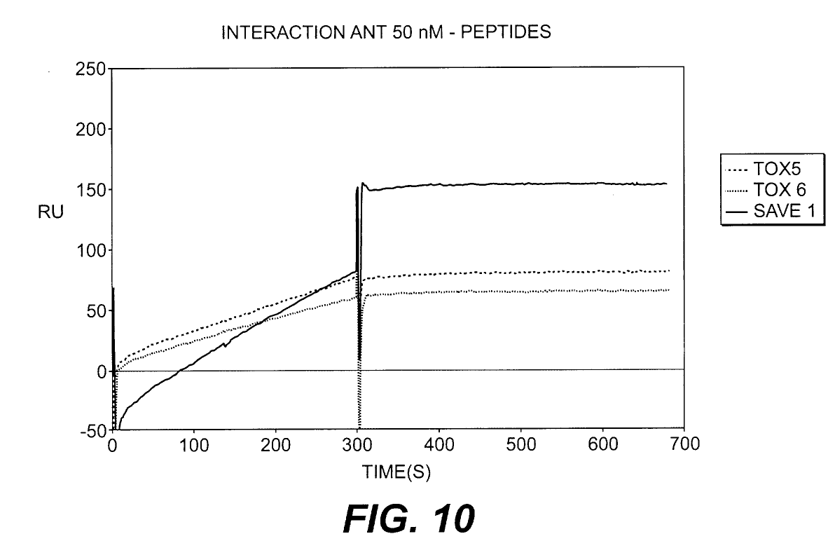

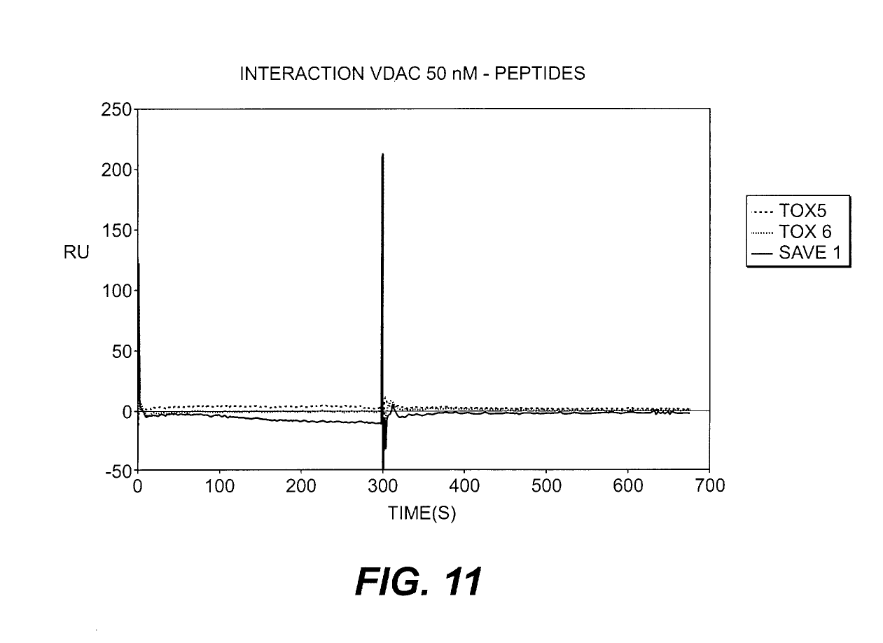

Thee other peptides were tested for their interaction with ANT at a concentration of 50 nM (FIG. 10). Purified ANT recognised Tox5, Tox6, and Save1 with relative affinities of respectively 0.1, 0.7, and 0.01 nM. These value being obtained at only one ANT concentration only give the relative affinity of ANT for the three peptides. Again, the use of 50 nM VDAC to interact with the same peptides did not result in any specific binding as shown in FIG. 11.

The following references have been cited herein. The entire disclosure of each reference cited herein is relied upon and incorporated by reference herein.

1—D. Decaudin, I. Marzo, C. Brenner and G. Kroemer. Mitochondria in chemotherapy-induced apoptosis: A prospective novel target of cancer therapy. Int J Oncol, 12: 141-52, 1998.

2—S. Fulda, S. A. Susin, G. Kroemer and K. M. Debatin. Molecular ordering of apoptosis induced by anticancer drugs in neuroblastorna cells. Cancer Res, 58: 4453-60, 1998a.

3—S. Fulda, C. Scaffidi, S. A. Susin, P. H. Krammer, G. Kroemer, M. E. Peter and K. M. 4-Debatin. Activation of mitochondria and release of mitochondrial apoptogenic factors by betulinic acid. J Biol Chem, 273: 33942-8, 1998b.

4—L. Ravagnan, I. Marzo, P. Costantini, S. A. Susin, N. Zamzami, P. X. Petit, F. Hirsch, M. Goulbern, M. F. Poupon, L. Miccoli, Z. Xie, J. C. Reed and G. Kroemer. Lonidamine triggers apoptosis via a direct, Bcl-2-inhibited effect on the mitochondrial permeability transition pore. Oncogene, 18: 2537-46, 1999.

5—N. Larochette, D. Decaudin, E. Jacotot, C. Brenner, I. Marzo, S. A. Susin, N. Zamzami, Z. Xie, J. Reed and G. Kroemer. Arsenite induces apoptosis via a direct effect on the mitochondrial permeability transition pore. Exp Cell Res, 249: 413-21, 1999.

6—P. Marchetti, N. Zamzami, B. Joseph, S. Schraen-Maschke, C. Mereau-Richard, P. Costantini, D. Metivier, S. A. Susin, G. Kroemer and P. Formstecher. The novel retinoid 6-[3-(1-adamantyl)-4-hydroxyphenyl]-2-naphtalene carboxylic acid can trigger apoptosis through a mitochondrial pathway independent of the nucleus. Cancer Res, 59: 6257-66, 1999.

7—P. Costantini, E. Jacotot, D. Decaudin and G. Kroemer. Mitochondrion as a novel target of anticancer chemotherapy. J. Natl. Cancer Inst., 92: 1042-1053, 2000.

8—1. Marzo, C. Brenner, N. Zamzami, S. A. Susin, G. Beutner, D. Brdiczka, R. Remy, Z. H. Xie, J. C. Reed and G. Kroemer. The permeability transition pore complex: a target for apoptosis regulation by caspases and Bcl-2-related proteins. J Exp Med, 187: 1261-71, 1998a.

9—I. Marzo, C. Brenner, N. Zamzami, J. M. Jurgensmeier, S. A. Susin, H. L. A. Vieira, M. C. Prévost, Z. Xie, S. Matsuyama, J. C. Reed and G. Kroemer. Bax and Adenine Nucleotide Translocator Cooperate in the Mitochondrial Control of Apoptosis. Science, 281: 2027-2031, 1998b.

10—Y. Tsujimoto, L. R. Finger, J. Yunis, P. C. Nowell and C. M. Croce. Cloning of the chromosome breakpoint of neoplastic B cells with the t(14;18) chromosome translocation. Science, 226: 1097-9, 1984.

11—J. C. Reed, M. Cuddy, T. Slabiak, C. M. Croce and P. C. Nowell. Oncogenic potential of Bcl-2 demonstrated by gene transfer. Nature, 336: 259-61, 1988.

12—M. Narita, S. Shimizu, T. Ito, T. Chittenden, R. J. Lutz, H. Matsuda and Y. Tsujimoto. Bax interacts with the permeability transition pore to induce permeability transition and cytochrome c release in isolated mitochondria. Proc Natl Acad Sci U S A, 95: 14681-6, 1998.

13—C. Brenner, H. Cadiou, H. L. Vieira, N. Zamzami, 1. Marzo, Z. Xie, B. Leber, D. Andrews, H. Duclohier, J. C. Reed and G. Kroemer. Bcl-2 and Bax regulate the channel activity of the mitochondrial adenine nucleotide translocator. Oncogene, 19: 329-36, 2000.

14—E. Jacotot, L. Ravagnan, M. Loeffler, K. F. Ferri, H. L. Vieira, N. Zamzami, P. Costantini, S. Druillennec, J. Hoebeke, J. P. Briand, T. lrinopoulou, E. Daugas, S. A. Susin, D. Cointe, Z. H. Xie, J. C. Reed, B. P. Roques and G. Kroemer. The HIV-1 viral protein R induces apoptosis via a direct effect on the mitochondrial permeability transition pore. J Exp Med, 191: 33-46, 2000.

15—P. Costantini, A. S. Belzacq, H. L. Vieira, N. Larochette, M. A. de Pablo, N. Zamzami, S. A. Susin, C. Brenner and G. Kroemer. Oxidation of a critical thiol residue of the adenine nucleotide translocator enforces Bcl-2-independent permeability transition pore opening and apoptosis. Oncogene, 19: 307-14, 2000.

16—S. Shimizu, A. Konishi, T. Kodama and Y. Tsujimoto. BH4 domain of antiapoptotic Bcl-2 family members closes voltage-dependent anion channel and inhibits apoptotic mitochondrial changes and cell death. Proc Natl Acad Sci U S A, 97: 3100-5, 2000.

17—M. G. VanderHeiden, N. S. Chandel, P. T. Schumacker and C. B. Thompson. Bcl-x(_{L}) prevents cell death following growth factor withdrawal by facilitating mitochondrial ATP/ADP exchange. Molecular Cell, 3: 159-167, 1999.

18—Griffioen, A. W., Molema, G. (2000). Angiogenesis: Potentials for Pharmacologic Intervention in the Treatment of Cancer, Cardiovascular Diseases, and Chronic Inflammation. Pharmacological Reviews 52:237-268.

What is claimed is:

1. Method for inducing or preventing the apoptosis of.eukaryotic cells comprising the homing on specific tissue cell population of a chimeric bifunctional molecule able to modulate-the activity of permeability transition pore complex (PTPC).

2. A method according to claim 1, wherein said chimeric molecules modulate the activity of the permeability transition pore complex (PTPC) of a specific eukaryotic cell by the regulation of opening or the closing of said pore.

3. A method according to claim 1 or 2, wherein said chimeric molecules comprising at least a first functional molecule and a second functional molecule, wherein said first functional molecule has the function to target specifically a tissue cell population, and the second functional molecule has the function to regulate the apoptosis activity linked to the permeability transition pore complex (PTPC) of said specific calls.

4. A method according to claim 3, wherein said chimeric molecules comprising at least a first functional molecule and a second functional molecule, wherein said first functional molecule has the function to target and to enter specifically in a tissue cell population and the second functional molecule has the function to regulate the apoptosis activity linked to the permeability transition pore complex (PTPC) of said specific cells.

5. A method according to claim 3, wherein said chimeric molecules comprising at least a first functional molecule and a second functional molecule, wherein said first functional molecule has the function to target and to enter specifically in a tissue cell population of interest and the second functional molecule has the function to target specifically and inducing or preventing the death of said cells by apoptosis by the regulation of the opening or the closing of the permeability transition pore complex (PTPC) of mitochondria or a fragment thereof.

6. A method according to claim 4, wherein said chimeric molecule has the formula:
Targ-Tox,
wherein Tox is a viral or a retroviral apoptotic peptide or a peptidomimetic or a fragment of a protein that interacts with permeability transition pore complex (PTPC) of a specific eukaryotic cell to cause apoptosis of the cell; and Targ is chosen from:
an antibody,
an antibody fragment,
arecombinant antibody fragment,
M350/ScFv,
V461/ScFv,
a homing peptide, and
any peptide chosen in Table III,
wherein said molecule binds and enters the cell specifically.

7. A method according to claim 5, wherein said chimeric molecule has the formula
Targ-Save,
wherein Save is a viral or a retroviral or a cellular antiapoptotic peptide or peptidomimetic or a fragment of protein that interacts with permeability transition pore complex (PTPC) of a specific eukaryotic cell to prevent the apoptosis of the cell with the proviso that when Save peptide is a viral peptide, Save is not vMIA protein of Cytomegalovirus; and
Targ is chosen from:
an antibody,
an antibody fragment,
a recombinant antibody fragment,
M350/ScFv,
a homing peptide, and
any peptide chosen in Table III,
wherein said molecule binds and enters the cell specifically.

8. A method according to anyone of claims 1 to 7, wherein said chimeric molecules comprises a Mitochondrial Localisation Sequence (MLS), which has the function to address specifically the second functional molecule to mitochondrial or intermembrane space—of the mitochondria.

9. A method according to claims 1, 2, 3, 4, 5, 6 and 8, wherein Tox is chosen from the group of peptides of Table I.

10. A method according to claims 1, 2, 3, 4, 5, and 7, wherein Save is chosen from the group of peptides of Table II.

11. A method according to any one of claims 1 to 10, wherein the second functional molecule of said chimeric molecules has the function to interact specifically with ANT of the PTPC of mitochondria also refers to as adenine nucleotide translocator isoforms 1, 2, or 3.

12. A chimeric bifunctional molecule capable to enter specifically in a tissue cell population for induce or prevent death of said cell by apoptosis and comprising at least a first functional molecule covalently linked to a second functional molecule, wherein said first functional molecule has the function to target and to enter specifically in a tissue cell population of interest and the second functional molecule has the function to target specifically and inducing or preventing the death of said cells by apoptosis by the regulation of the opening or the closing of the permeability transition pore complex (PTPC) of mitochondria or a fragment thereof.

13. A chimeric molecule according to claim 12 which has the formula:
Targ-Tox,
wherein Tox is a viral or a retroviral apoptotic peptide or peptidomimetic or a fragment of a protein that interacts with PermeabilityTransifion Pore Complex (PTPC) of a specific eukaryofic cell to cause apoptosis of the cell; and
Targ is chosen from:
an antibody,
an antibody fragment,
a recombinant antibody fragment,
M350/ScFv,
V461/ScFv,
a homing peptide, and
any peptide of Table III,
wherein said molecule binds and enters the cell specifically.

14. A chimeric molecule according to claim 12 which has of the formula
Targ-Save
Wherein Save is a viral or a retroviral or a cellular antiapoptotic peptide or peptidomimetic or a fragment of protein that interacts with Permeability Transition. Pore Complex (PTPC) of a specific eukaryotic cell to prevent apoptosis of the cell, with the proviso that when Save peptide is a viral peptide, Save is not vMIA protein of Cytomegalovirus;
and Targ is chosen from:
an antibody,
an antibody fragment,
a recombinant antibody fragment,
M350/ScFv,
a homing peptide, and
any peptide of Table III,
wherein said molecule binds and enters the cell specifically.

15. A chimeric molecule according to any of claims 12 to 14 comprising a mitochondrial localisation sequence (MLS) which has the function to address specifically the second functional molecule to mitochondrial membranes or intermembrane space.

16. A chimeric molecule according to claims 13 or 15, wherein Tox is chosen from the group of peptides of Table I.

17. A chimeric molecule according to claims 14 and 15, wherein wherein Save is chosen from the group of peptides of Table II.

18. A chimeric molecule according to claims 13, 15 and 16, wherein the Targ and Tox peptides are covalently bonded through a peptide linker comprising 3 to 18 amino acids.

19. A chimeric molecule according to claims 14, 15 and 17, wherein the Targ and Save peptides are covalently bonded through a peptide linker comprising 3 to 18 amino acids.

20. A vector encoding a chimeric molecule as claimed in any one of claims 12 to 19.

21. A hybridoma secreting Targ according to claim 13 or 14 and deposited at the National Collection of Culture and Microorganism (C.N.C.M.) on Jan. 24, 2001, under the accession number n° I 2617.

22. A purified monoclonal antibody encoded by the hybridoma of claim 21.

23. A recombinant host cell comprising a vector as claimed in claim 20.

24. A cancer cell having a tumor associated antigen on the surface thereof to which is bound the chimeric molecule as claimed in any one of claims 12 to 19.

25. A method of determining the presence of a cancer cell having a tumor-associated antigen on the surface thereof in a biological sample comprising:
a) contacting a biological sample of interest with a chimeric peptide molecule according to claims 12 to 19 under conditions to permit the binding between the chimeric peptide according to the invention and the antigen on the surface of the. cancer cell,
b) detecting the binding by usual technique; and
c) optionally quantifying the binding detected in step b).

26. A method for inducing death by apoptosis in a tumoral or viral infected cell having a tumor-associated antigen on surface thereof in a biological sample comprising:
contacting a biological sample of interest with a chimeric peptide molecule according to claims 16 or 17 under conditions to permit the binding between the chimeric peptide according to the invention and the antigen on the surface of the cancer cell and for a time sufficient to allow the entry inside the cell and death cell by apoptosis or viral infected cells.

27. A method for prevent cell death by mitochondrial apoptosis comprising contacting a biological sample of interest with a chimeric molecule, molecule according to claims 17 or 19 under conditions to permit the binding between the chimeric molecule according to the invention and the cell of interest and for a time sufficient to allow the entry inside cell of interest and prevent the cell death by apoptosis.

28. A method for prevent cell death according to claim 27, wherein the cells of interest are choosen among the following cell populations: neurons, cardiocytes, and hepatocytes.

29. A method for identifying an active agent of interest that interacts with the activity of the permeability transition pore complex (PTPC) comprising
a) contacting a biological sample containing cells with permeability transition pore complex (PTPC) with a chimeric peptide according to claims 12 to 19 in the presence of a candidate agent; and
b) comparing the binding of the chimeric peptide with the permeability transition pore complex (PTPC) in absence of said agent.
c) optionally, testing the activity of said selected agent on a preparation of a cellular extract comprising subcellular elements with the permeability transition pore complex (PTPC).

30. A method for identifying an active agent of interest that interacts with ANT peptide of permeability transition pore complex (PTPC) comprising:
d) contacting a biological sample containing cells with ANT peptide of permeability transition pore complex (PTPC) with a chimeric peptide according to claims 12 to 19 in the presence of a candidate agent; and
e) comparing the binding of the chimeric peptide with the ANT peptide of the permeability transition pore complex (PTPC) in absence of said agent.
f) optionally, testing the activity of said selected agent on a preparation of a cellular extract comprising subcellular elements with the ANT peptide of the permeability transition pore complex (PTPC).

31. A method of identification of mitochondrial antigen, said antigen having the capacity to interact with a macromolecule or a molecule or a peptide carrying the characteristic of Tox according to claims 13 or 16.

32. A method of identification of mitochondrial antigen, said antigen having the capacity to interact with a macromolecule or a molecule or a peptide carrying the characteristic of save according to claims 14 or 17.

33. A method of treatment or of prevention of a pathological infection or disease comprising the administration to a patient of the pharmaceutical composition containing at least a chimeric molecule according to any of claims 12 to 19.

34. A pharmaceutical composition comprising at least a chimeric molecule according to claims any of 12 to 19.
